(19) United States
(12) Patent Application Publication
(10) Pub. No.: US 2008/0318929 A1
(43) Pub. Date: Dec. 25, 2008

(54) Pyrimidine Derivatives Useful as Inhibitors of Pkc-Theta

(76) Inventors: Antonio J.M. Barbosa, Middlebury, CT (US); Steven Richard Brunette, New Milford, CT (US); Eugene Richard Hickey, Danbury, CT (US); Michael David Lawlor, Seymour, CT (US); Matt Aaron Tschantz, Newtown, CT (US)

Correspondence Address: MICHAEL P. MORRIS;BOEHRINGER INGELHEIM USA CORPORATION, 900 RIDGEBURY ROAD, P O BOX 368, RIDGEFIELD, CT, 06877-0368, US

(21) Appl. No.: 12/158,217

(22) PCT Filed: Dec. 12, 2006
(86) PCT No.: PCT/US06/61899

§ 371 Date: Jun. 26, 2008

Related U.S. Application Data
(60) Provisional application No. 60/743,066, filed on Dec. 21, 2005.

(51) Int. Cl. A61K 31/506; A61P 37/00; A61P 29/00; A61P 3/10; C07D 239/50; C07D 401/12; A61K 31/5377; A61K 31/55; C07D 401/10; C07D 487/04; C07D 403/12; A61K 31/505

(52) U.S. Cl. 514/215; 514/275; 544/323; 544/324; 540/578; 544/295; 514/252.14; 514/235.8

(57) ABSTRACT

Disclosed are novel compounds of formula (I) wherein R₁, R₂, R₃, and R₄ and A are as defined herein, which are useful as inhibitors of PKC-theta and are thus useful for treating a variety of diseases and disorders that are mediated or sustained through the activity of PKC-theta, including immunological disorders and type II diabetes. This invention also relates to pharmaceutical compositions comprising these compounds, methods of using these compounds in the treatment of various diseases and disorders, processes for preparing these compounds and intermediates useful in these processes.

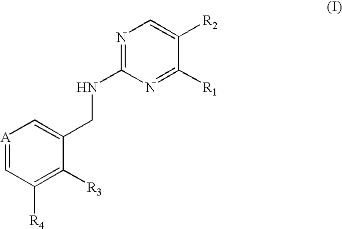

### CROSS-REFERENCE TO RELATED APPLICATIONS

This application claims the benefit of U.S. Provisional Application No. 60/743,066, filed Dec. 21, 2005.

### FIELD OF THE INVENTION

This invention relates to substituted pyrimidine derivatives which are useful as inhibitors of PKC-theta and are thus useful for treating a variety of diseases and disorders that are mediated or sustained through the activity of PKC-theta, including immunological disorders and type II diabetes. This invention also relates to pharmaceutical compositions comprising these compounds, methods of using these compounds in the treatment of various diseases and disorders, processes for preparing these compounds and intermediates useful in these processes.

### BACKGROUND OF THE INVENTION

The protein kinase C family is a group of serine/threonine kinases that is comprised of twelve related isoenzymes. These kinases are expressed in a wide range of tissues and cell types. Its members are encoded by different genes and are sub-classified according to their requirements for activation. The classical PKC enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKC-theta is a member of the nPKC sub-family. It has a restricted expression pattern, found predominantly in T cells and skeletal muscle. Upon T cell activation, a supramolecular activation complex (SMAC) forms at the site of contact between the T cell and antigen presenting cell (APC). PKC-theta is the only PKC isoform found to localize at the SMAC(C. Monks et al., Nature, 1997, 385, 83), placing it in proximity with other signaling enzymes that mediate T cell activation processes. In another study (G. Baier-Bitterlich et al., Mol. Cell. Biol., 1996, 16, 842) the role of PKC-theta in the activation of AP-1, a transcription factor important in the activation of the IL-2 gene, was confirmed. In unstimulated T cells, constitutively active PKC-theta stimulated AP-1 activity while in cells with dominant negative PKC-theta, AP-1 activity was not induced upon activation by PMA. Other studies showed that PKC-theta, via activation of IκB kinase beta, mediates activation of NF-κB induced by T cell receptor/CD28 co-stimulation (N. Coudronniere et al., Proc. Nat. Acad. Sci. U.S.A., 2000, 97, 3394; X. Lin et al., Moll. Cell. Biol., 2000, 20, 2933). Proliferation of peripheral T cells from PKC-theta knockout mice, in response to T cell receptor (TCR)/CD28 stimulation was greatly diminished compared to T cells from wild type mice. In addition, the amount of IL-2 released from the T cells was also greatly reduced (Z. Sun et al., Nature, 2000, 404, 402). Otherwise, the PKC-theta knockout mice seemed normal and were fertile.

The studies cited above and other studies confirm the critical role of PKC-theta in T cell activation and subsequent release of cytokincs such as IL-2 and T cell proliferation (A. Altman et al., Immunology Today, 2000, 21, 567). Thus an inhibitor of PKC-theta would be of therapeutic benefit in treating immunological disorders and other diseases mediated by the inappropriate activation of T cells.

It has been well established that T cells play an important role in regulating the immune response (Powrie and Coffman, Immunology Today, 1993, 14, 270). Indeed, activation of T cells is often the initiating event in immunological disorders. Following activation of the TCR, there is an influx of calcium that is required for T cell activation. Upon activation, T cells produce cytokines, including IL-2, leading to T cell proliferation, differentiation, and effector function. Clinical studies with inhibitors of IL-2 have shown that interference with T cell activation and proliferation effectively suppresses immune response in vivo (Waldmann, Immunology Today, 1993, 14, 264). Accordingly, agents that inhibit T lymphocyte activation and subsequent cytokine production are therapeutically useful for selectively suppressing the immune response in a patient in need of such immunosuppression and therefore are useful in treating immunological disorders such as autoimmune and inflammatory diseases.

In addition, PKC-theta activation has been shown to be associated with insulin resistance in skeletal muscle (M. E. Griffen et al., Diabetes, 1999, 48, 1270). Therefore inhibitors of PKC-theta may also be useful for treating type II diabetes.

Bornemann et al., US Publication No. 2003/0134838 A1 discloses trisubstituted pyrimidines as beta.-amyloid modulators. Dahmann et al, US Publication No. 2003/0171359 A1 discloses trisubstituted pyrimidines for the treatment of illnesses characterised by excessive or abnormal cell proliferation. Cardozo et al, U.S. Publication Nos. 2004/0242613 A1 and 2005/0124640 A1 disclose 2,4-diaminopyrimidine derivatives as inhibitors of PKC-theta. WO 03/106451 discloses certain substituted diaminopyrimidine compounds as inhibitors of PKC-theta. WO 04/065378 discloses certain 2-aminopyridine compounds as cyclin-dependent kinase 4 (CDK/4) inhibitors useful in the treatment of cell proliferative diseases. WO 04/011456 discloses certain substituted 2,4-diaminopyridine compounds as protein tyrosine kinase inhibitors.

There is a continuing need in the art for compounds that are potent and selective inhibitors of PKC-theta.

### BRIEF SUMMARY OF THE INVENTION

In a general aspect, the present invention is directed to the compounds of the following formula (I):

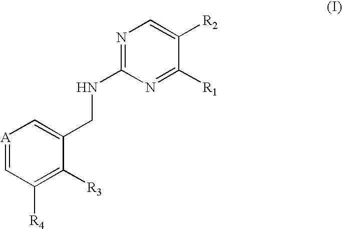

wherein R₁, R₂, R₃, R₄ and A are as defined herein, as well as the tautomers, pharmaceutically acceptable salts, solvates, and amino-protected derivatives thereof. It has been found that the compounds of formula (I) have valuable pharmacological properties, particularly an inhibiting activity on PKC-theta. Many of the compounds of the invention are not only potent inhibitors of PKC-theta but are also selective for the inhibition of PKC-theta as compared to one or more other protein kinases.

In another aspect, the present invention is directed to a method of inhibiting PKC-theta activity in a patient comprising administering to the patient a compound of the present invention as described above.

In another aspect, the present invention is directed to a method of treating a disease or disorder associated with the activation of T cells in a patient comprising administering to the patient a therapeutically effective amount of a compound of the present invention as described above.

In another aspect, the present invention is directed to a method of treating an immunological disorder in a patient comprising administering to the patient a therapeutically effective amount of a compound of the present invention as described above. Examples of such immunological disorders that may be treated include, for example, inflammatory diseases, autoimmune diseases, organ and bone marrow transplant rejection and other disorders associated with T cell mediated immune response, including acute or chronic inflammation, allergies, contact dermatitis, psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection) and lupus erythematosus.

In another aspect, the present invention is directed to a method of treating type II diabetes in a patient comprising administering to the patient a therapeutically effective amount of compound of the present invention as described above.

In yet additional aspects, the present invention is directed to pharmaceutical compositions comprising the above-mentioned compounds, processes for preparing the above-mentioned compounds and intermediates used in these processes.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the present specification and claims, however, unless specified to the contrary, the following terms have the meaning indicated and the following conventions are adhered to.

### A. Chemical Nomenclature, Terms, and Conventions

In general, for groups comprising two or more subgroups, the last named group is the radical attachment point, for example, “alkylaryl” means a monovalent radical of the formula Alk-Ar—, while “arylalkyl” means a monovalent radical of the formula Ar-Alk-(where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

All references to a chemical group being “substituted with” another chemical group shall be understood to mean the first chemical group can be substituted with one or more of the second chemical group, with the exception of any substitution pattern that is not physically or chemically possible or results in a unstable structure or compound. For example, the phrase “C₁₋₆ alkyl, which is optionally substituted with halogen” shall mean a C₁₋₆ alkyl group having one or multiple halogen substituents being the same or different from each other. All alkyl groups shall be understood as being branched or unbranched unless otherwise specified. Other more specific definitions are as follows:

The term “heteroaryl” refers to a stable 5 or 6 membered, monocyclic aromatic heterocycle radical, wherein the heterocycle radical is optionally fused to either an aryl, e.g. benzene, or to a second 5 or 6 membered, monocyclic aromatic heterocycle to form in each case a bicyclic heteroaryl group. Each heterocycle consists of carbon atoms and from 1 to 3 heteroatoms chosen from nitrogen, oxygen and sulfur. The heterocycle may be attached by any atom of the cycle, which results in the creation of a stable structure. Example “heteroaryl” radicals include, pyridyl, pyrimidinyl, pyrazinyl, pyridazinyl, pyrrolyl, imidazolyl, pyrazolyl, thienyl, furyl, isoxazolyl, isothiazolyl, oxazolyl, thiazolyl, oxadiazolyl, thiadiazolyl, quinolinyl, isoquinolinyl, indolyl, benzimidazolyl, benzofuranyl, benzoxazolyl, benzisoxazolyl, benzopyrazolyl, benzothiofuranyl, benzothiazolyl, quinazolinyl and indazolyl.

The term “aryl” shall be understood to mean a 6-10 membered monocyclic or bicyclic aromatic carbocycle, and includes, for example, phenyl and naphthyl; other terms comprising “aryl” will have the same definition for the aryl component, and examples of these moieties include: arylalkyl, aryloxy or arylthio.

The term “oxo” refers to a double-bonded oxygen group (═O).

The term “amino protected derivatives” shall be understood to mean compounds of formula (I) wherein one or more of the amine groups are protected by suitable amino protecting groups. Amino protecting groups that may be used include, for example, alkoxycarbonyl groups, such as tert-butyloxycarbonyl (Boc) and ethoxycarbonyl, Mannich bases, Schiff bases and amino acids. As would be understood by a person skilled in the art, such amino protected compounds may be useful as intermediates in the preparation of other compounds of formula (I), e.g., as described in the synthetic processes below, and/or may themselves be useful as prodrugs that can be administered to a patient to be converted in vivo into a PKC-theta inhibitor having the resulting pharmacologic and therapeutic effects expected from the inhibition of PKC-theta in a patient.

The term “pharmaceutically acceptable salts” include those derived from pharmaceutically acceptable inorganic and organic acids and bases. Examples of suitable acids include hydrochloric, hydrobromic, carbonic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids, such as oxalic acid, while not themselves pharmaceutically acceptable, may be employed in the preparation of salts useful as intermediates in obtaining the compounds of this invention and their pharmaceutically acceptable acid addition salts. Salts derived from appropriate bases include alkali metal (e.g., sodium), alkaline earth metal (e.g., magnesium), ammonium and N—(C₁₋₄ alkyl)₄⁺ salts.

The term “solvate” means a physical association of a compound with one or more solvent molecules or a complex of variable stoichiometry formed by a solute (for example, a compound of Formula (I)) and a solvent, for example, water, EtOH, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompasses both solution-phase and isolatable solvates. Representative solvates include hydrates, EtOHatcs, McOHates, and the like. The term “hydrate” means a solvate wherein the solvent molecule(s) is/are H₂O.

The term “compounds of the invention” and equivalent expressions are meant to embrace compounds of Formula (I) as herein described, including the tautomers, pharmaceutically acceptable salts, solvates, and amino-protected derivatives thereof, where the context so permits. In general, the compounds of the invention and the formulas designating the compounds of the invention are understood to only include the stable compounds thereof and exclude unstable compounds, even if an unstable compound might be considered to be literally embraced by the compound formula.

The term “stable compound” means a compound that is sufficiently robust to survive isolation to a useful degree of purity from a reaction mixture, and formulation into an efficacious therapeutic agent. For example, a compound which would have a “dangling valency” is not a compound contemplated by the invention.

Specific compounds of the present invention may be identified in the present specification by chemical name and/or chemical structure. In the event of any conflict between the chemical name and chemical structure, the chemical structure will control.

### B. Isomer Terms and Conventions

In general, all tautomeric and isomeric forms and mixtures thereof, for example, individual geometric isomers, stereoisomers, enantiomers, diastereomers, racemates, racemic or non-racemic mixtures of stereoisomers, mixtures of diastereomers, or mixtures of any of the foregoing forms of a chemical structure or compound is intended, unless the specific stereochemistry or isomeric form is specifically indicated in the compound name or structure.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the present invention from this disclosure and the knowledge in the art.

Preparation of Pure Stereoisomers, e.g. Enantiomers and Diastereomers, or Mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

### C. Pharmaceutical Administration Terms and Conventions

The term “patient” includes both human and non-human mammals.

The term “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the state of the art, and this disclosure.

The phrase “disease or disorder associated with the activation of T cells” and similar expressions mean that the activation of T cells is a contributing factor to either the origin or continuation of the disease or disorder in the patient.

### EMBODIMENTS OF THE INVENTION

In its broadest generic aspect the invention provides novel compounds of formula (I) as described below:

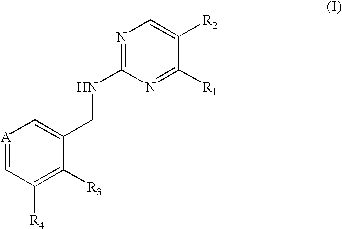

R₁ is selected from the following groups:

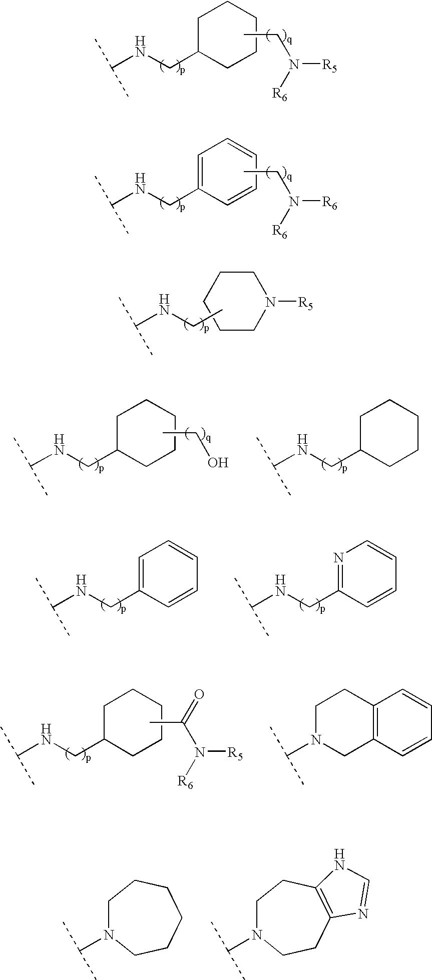

wherein:
p is 1, 2 or 3;
q is 0 or 1,
R₅, R₆ are each independently selected from:
(A) hydrogen,
(B) C₁₋₆alkyl, or wherein R₅ and R₆ together constitute a methylene bridge which together with the nitrogen atom between them forms a four to six-membered ring wherein one of the methylene groups is optionally replaced by an oxygen or nitrogen atom, and which ring is optionally and independently substituted by one or more of the following groups:
   (i) C₁₋₆alkyl
   (ii) COR₇, wherein R₇ is:
      (a) C₁₋₆alkyl,
      (b) C₁₋₆alkyloxy,
   (C) C₁₋₆alkylcarbonyl,
   (D) C₁₋₆alkylsulfonyl,
   (E) —CONR₈R₉, wherein R₈ and R₉ are each independently selected from:
      (i) hydrogen
      (ii) C₁₋₆alkyl;
         R₂ is selected from the following groups:
(F) CF₃,
(G) cyano,
(H) CONH₂
(I) halogen, or
(J) nitro;
   R₃ is selected from the following groups:
(A) hydrogen,
(B) C₁₋₆alkyl, which is optionally substituted with halogen,
(C) C₁₋₆alkyloxy, which is optionally substituted with halogen,
(D) halogen,
   R₄ is selected from the following groups:
(A) heteroaryl, which is optionally substituted with C₁₋₆alkyl;
(B) aryl or heteroaryl, which is substituted with one or more of the following groups:
   (i) C₁₋₆alkyl, which is substituted with hydroxyl, oxo, or NR₁₀R₁₁, wherein R₁₀ and R₁₁ are each independently selected from the following groups:
      (a) hydrogen,
      (b) C₁₋₆alkyl, which is optionally substituted with hydroxyl or CONH₂,
      (c) C₁₋₆alkylcarbonyl, which is optionally substituted with one or more halogens,
      (d) C₁₋₆alkylsulfonyl,
      (e) or wherein R₁₀ and R₁₁ constitute a methylene bridge which together with the nitrogen atom between them forms a four to six-membered ring,
   (ii) CONR₁₂R₁₃, wherein R₁₂ and R₁₃ are each independently selected from hydrogen or C₁₋₆alkyl,
   (iii) SO₂NR₁₂R₁₃, wherein R₁₂ and R₁₃ are each independently selected from hydrogen or C₁₋₆alkyl,
(C) —NR₁₄R₁₅, wherein R₁₄ and R₁₅ are each independently selected from:
   (i) C₁₋₆alkylcarbonyl, which is substituted with amino,
   (ii) or wherein R₁₄ and R₁₅ constitute a methylene bridge which together with the nitrogen atom between them forms a four to seven-membered ring, wherein one of the methylene groups is substituted with C₁₋₆alkyl, and wherein each C₁₋₆alkyl is optionally substituted with hydroxyl or NR₁₀R₁₁, wherein R₁₀ and R₁₁ are as defined previously,
(D)-CONR₁₆R₁₇, wherein R₁₆ and R₁₇ are each independently selected from:
   (i) C₁₋₆alkyl, which is substituted with hydroxyl or NR₁₈R₁₉, wherein R₁₈ and R₁₉ are each independently selected from hydrogen or C₁₋₆alkyl, or wherein R₁₈ and R₁₉ constitute a methylene bridge which together with the nitrogen atom between them forms a four to six-membered ring, wherein one of the methylene groups is optionally replaced by an oxygen;
(E) C₆alkynyl group optionally substituted by amino, C₁₋₃alkylamino, or di-(C₁₋₃alkyl)amino; and
   A is independently selected from carbon or nitrogen;
   or a tautomer, pharmaceutically acceptable salt, solvate or amino-protected derivative thereof.

In another embodiment there are provided compounds of formula (I) as described above and wherein: R₁ is selected from the following groups:

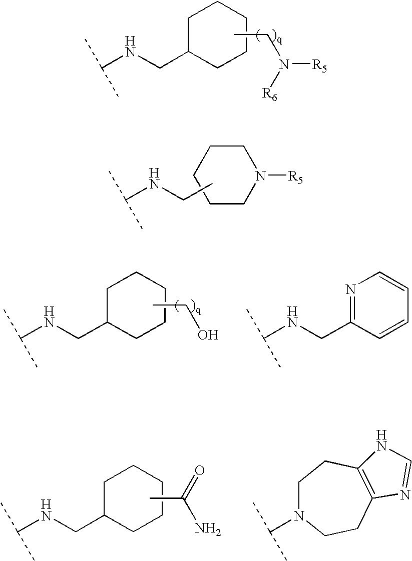

wherein:
q is 0 or 1,
R₅, R₆ are each independently selected from:
(A) hydrogen,
(B) or wherein R₅ and R₆ together constitute a methylene bridge which together with the nitrogen atom between them forms a five to six-membered ring wherein one of the methylene groups is optionally replaced by a nitrogen atom, and which ring is optionally and independently substituted by one or more of the following groups:
   (iv) C₁₋₆alkyl
   (v) COR₇, wherein R₇ is C₁₋₆alkyloxy,
(C) C₁₋₆alkylcarbonyl
(D) C₁₋₆alkylsulfonyl;
   R₂ is selected from the following groups:
(A) cyano, or
(B) nitro;
   R₃ is selected from the following groups:
(A) C₁₋₃alkyl,
(B) C₁₋₃alkyloxy, which is optionally substituted with fluorine,
(C) halogen;
   R₄ is selected from the following groups:
(A) aryl, which is substituted with one or more of the following groups:
   (i) C₁₋₃alkyl, which is substituted with hydroxyl or NR₂₀R₂₁, wherein R₂₀ and R₂₁ are each independently selected from the following groups:
      (f) hydrogen,
      (g) C₁₋₃alkyl, which is optionally substituted with hydroxyl or CONH₂,
      (h) or wherein R₂₀ and R₂₁ constitute a methylene bridge which together with the nitrogen atom between them forms a five to six-membered ring,
   (ii) CONH₂
   (iii) SO₂NH₂,
(B) 3-pyridyl, which is optionally substituted with C₁₋₃alkyl, wherein each alkyl group is optionally substituted with amino,
(C) —NR₂₂R₂₃, wherein R₂₂ and R₂₃ constitute a methylene bridge which together with the nitrogen atom between them forms a five to six-membered ring, wherein one of the methylene groups is substituted with C₁₋₃alkyl, and wherein each C₁₋₃alkyl is optionally substituted with OH or NR₂₀R₂₁, where R₂₀ and R₂₁ are as defined previously,
(D) —CONR₂₄R₂₅, wherein R₂₄ and R₂₅ are each independently selected from:
   (i) C₁₋₃alkyl, which is substituted with C₁₋₃alkylamino; and
      A is independently selected from carbon or nitrogen;
      or a tautomer, pharmaceutically acceptable salt, solvate or amino-protected derivative thereof.

In another embodiment there are provided compounds of formula (II) wherein:

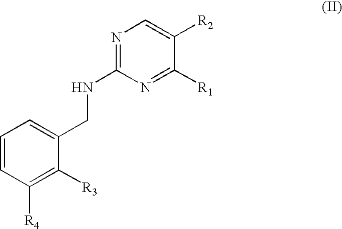

R₁ is selected from the following groups:

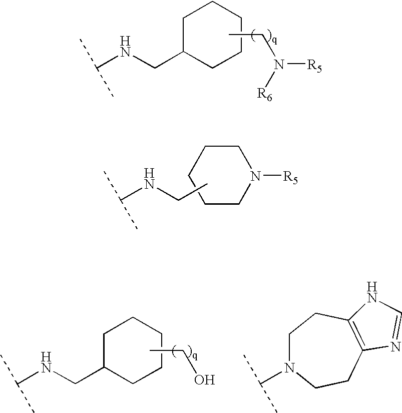

wherein:
q is 0 or 1
R₅, R₆ are each independently selected from:
(A) hydrogen,
(B) C₁₋₆alkylcarbonyl,
(C) C₁₋₆alkylsulfonyl;
   R₂ is selected from the following groups:
(A) cyano, or
(B) nitro;
   R₃ is selected from the following groups:
(A) CH₃,
(B) OCF₃,
(C) Cl;
   R₄ is selected from the following groups:

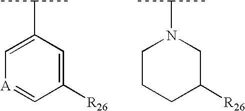

wherein:
R₂₆ is selected from the following groups:
(A) C₁₋₃alkyl, which is substituted with hydroxyl or NR₂₇R₂₈, wherein R₂₇ and R₂₈ are each independently selected from the following groups:
   (i) hydrogen,
   (ii) C₁₋₃alkyl, which is optionally substituted with hydroxyl or CONH₂,
(B) CONH₂
(C) SO₂NH₂; and
   A is carbon or nitrogen;
   or a tautomer, pharmaceutically acceptable salt, solvate or amino-protected derivative thereof.

### General Synthetic Methods

The compounds of the invention may be prepared by the methods described below. In each of the schemes below, the groups A, R₁, R₂, R₃, and R₄ are as defined above for general formula I unless noted otherwise. Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section. Typically, reaction progress may be monitored by thin layer chromatography (TLC) if desired. Intermediates and products may be purified by chromatography on silica gel and/or recrystallization. Starting materials and reagents are either commercially available or may be prepared by one skilled in the art from commercially available materials using methods described in the chemical literature.

Compounds of formula (I) having R₁═NR′R″ may be prepared as illustrated in Scheme I and described below.

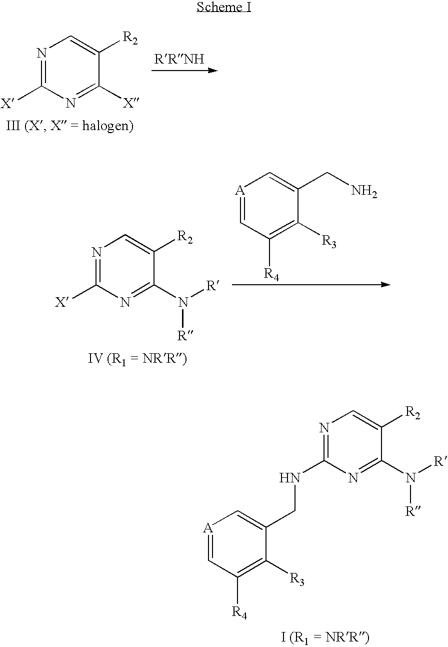

As illustrated above, a 2,4-dihalopyrimidine (III), preferably a 2,4-dichloropyrimidine, is reacted with about one equivalent of an amine (R′R″NH) in the presence of base, such as triethylamine, in a suitable solvent, such as EtOH or methylene chloride, to provide intermediate IV. The reaction is carried out preferably at about 0° C. to about room temperature. Intermediate IV is then reacted with a second amine ArCH₂NH₂ (Ar=a substituted phenyl or pyridyl) in a suitable solvent, such as methylene chloride, to provide a compound of formula (I). The reaction is carried out preferably at about room temperature.

For compounds of formula (I) where R₄ is substituted phenyl or optionally substituted pyridyl, the compounds may be prepared as illustrated in Scheme II and described below.

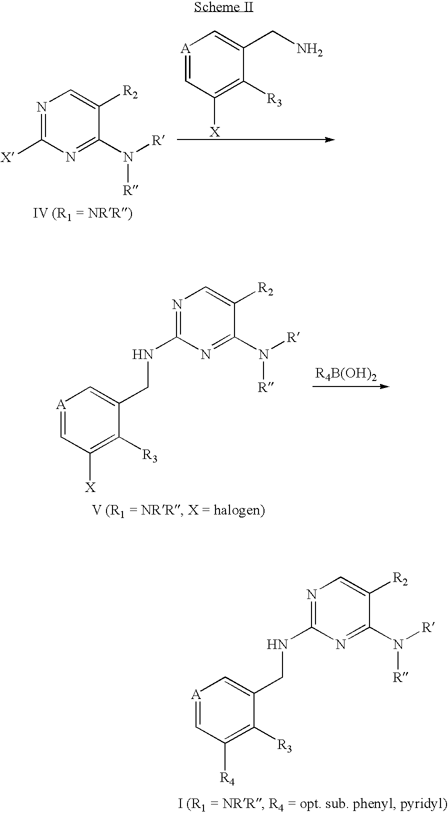

As illustrated above intermediate (IV) is reacted with an amine ArCH₂NH₂ (Ar=substituted phenyl or pyridyl, X=halogen) in a suitable solvent, such as methylene chloride, to provide intermediate V. The reaction is carried out preferably at about room temperature. Intermediate V is then reacted with a boronic acid R₄B(OH)₂, in a suitable solvent mixture, such as DME (dimethoxyethane) and water, and in the presence of a catalyst, such as tetrakis(triphenylphosphine)palladium, and a base, such as sodium carbonate, to provide the desired I. The reaction is preferably heated to about the reflux temperature of the solvent.

If R₁ contains a second amine group, (i.e., in the R′ and/or R″ groups in Scheme I and II above) the second amine is preferably protected with a suitable amino-protecting group, for example with a Boc-group, prior to reaction with intermediate III, and the amine is deprotected after reaction of the pyrimidine intermediate IV with ArCH₂NH₂, or after reaction of the pyrimidine intermediate V with boronic acid R₄B(OH)₂. For example, in the case of 1-amino-4-aminomethylcyclohexane as illustrated in Scheme III, the mono-Boc-protected diamine is reacted with (III) as described above. The resulting intermediate (VI) is then reacted with ArCH₂NH₂ as described above, and the Boc-protected intermediate (VII) is then deprotected by treatment with acid to provide the desired compound of formula (I).

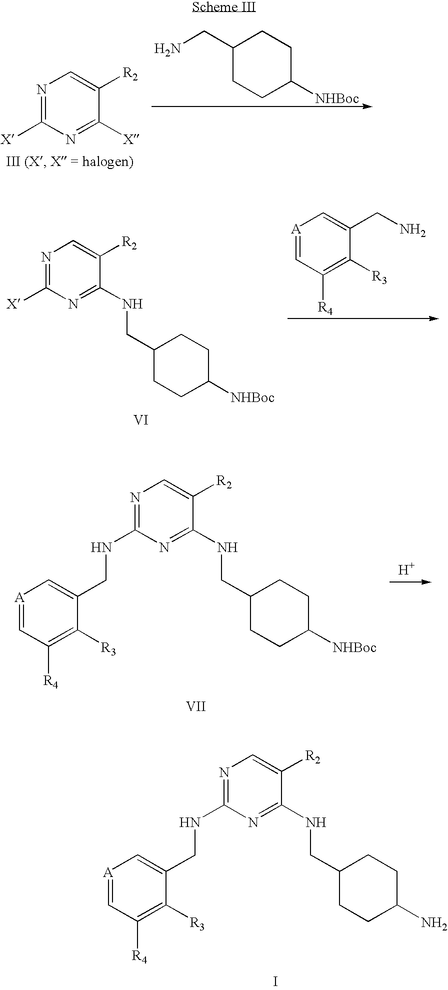

In a variation illustrated in Scheme IV, if R₂ is NO₂, intermediate (III) may be reacted with a thiocyanate salt, such as potassium thiocyanate, in a suitable solvent, such as EtOH, to produce VIII. Intermediate VIII is reacted with ArCH₂NH₂ in a suitable solvent, such as methylene chloride, and in the presence of base, such as triethylamine, to provide IX. Intermediate IX may then be reacted with an amine R′R″NH in a suitable solvent, such as methylene chloride or DMF, to provide the desired compound of formula (I).

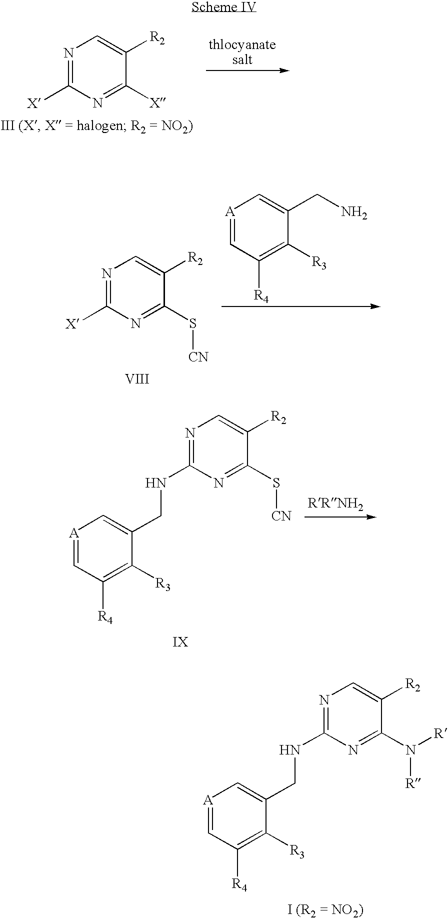

Substituents R₁, R₃, and R₄ may be further modified by methods known in the art to obtain additional examples of formula (I). Some of these modifications are illustrated in the synthetic examples below.

In order that this invention be more fully understood, the following examples are set forth. These examples are for the purpose of illustrating embodiments of this invention, and are not to be construed as limiting the scope of the invention in any way. Starting materials used are either commercially available or easily prepared from commercially available materials by those skilled in the art.

### SYNTHETIC EXAMPLES

### Example 1

### Synthesis of N²-{[3′-(aminomethyl)biphenyl-3-yl]methyl}-N⁴-{[trans-4-(aminomethyl)cyclohexyl]methyl}-5-nitropyrimidine-2,4-diamine

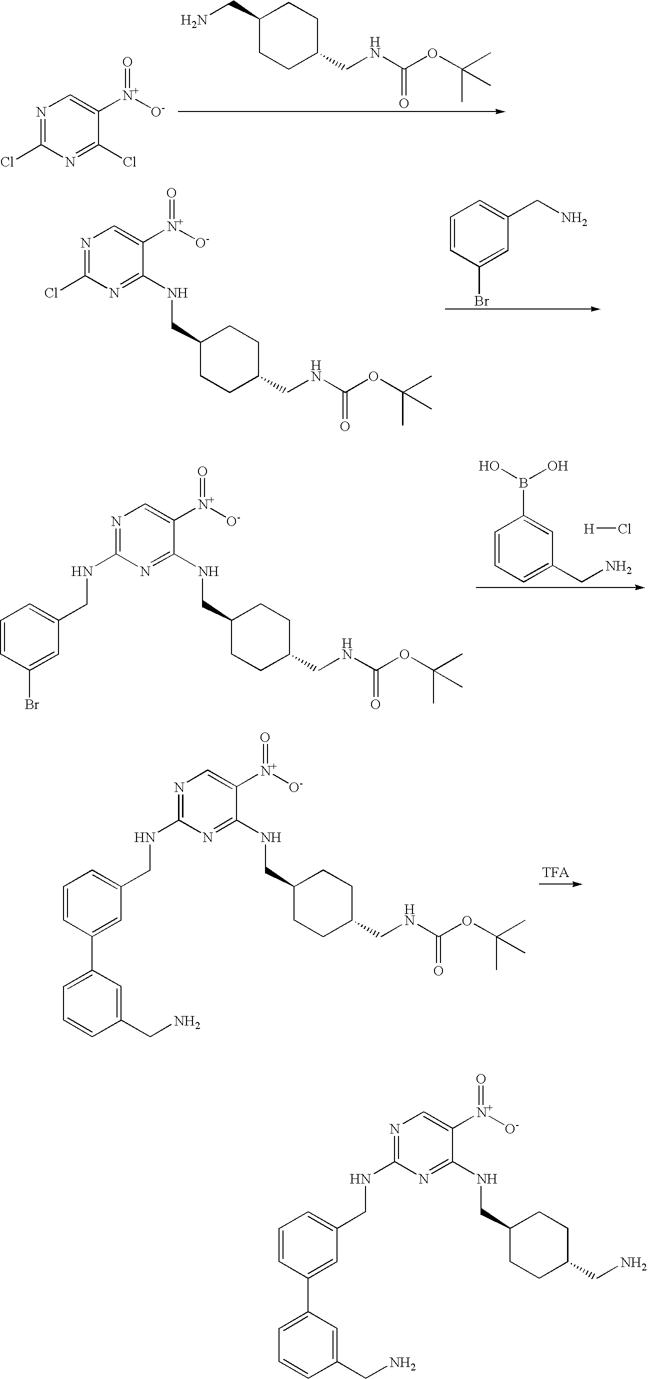

To a mixture of 2,6-dichloro-5-nitropyrimidine (17.13 g, 88.30 mmol) and CH₃CN (50 mL) at 0° C. was added a mixture of trans-(4-aminomethyl-cyclohexylmethyl)-carbamic acid tert-butyl ester (21.40 g, 88.30 mmol) and N,N-diisopropylethylamine (15.4 mL, 88.30 mmol) in CH₃CN (50 mL). The reaction mixture was allowed to warm to room temperature and stirred overnight. Volatiles were evaporated in vacuo and the residue purified by silica gel chromatography (Hexane/EtOAc 4:1) to afford trans-4-[(2-chloro-5-nitro-pyrimidin-4-ylamino)-methyl]-cyclohexylmethyl}-carbamic acid tert-butyl ester (25.00 g, 71%) as an off-white solid.

To a solution of 3-bromo-benzylamine (716 mg, 3.85 mmol) and diisopropylethylamine (0.65 mL, 3.75 mmol) in dichloromethane (25 mL) was added trans-4-[(2-chloro-5-nitro-pyrimidin-4-ylamino)-methyl]-cyclohexylmethyl}-carbamic acid tert-butyl ester (1.01 g, 2.53 mmol). The r×n mixture was stirred at room temperature for 17 h, then partitioned between ethyl acetate and 1M HCl solution. The organic phase was washed with satd NaHCO₃ solution and brine, dried over Na₂SO₄, filtered and concentrated. The crude product was purified by silica gel chromatography eluting with 0-3% MeOH in CH₂Cl₂ to afford 723 mg (52%) of (trans-4-{[2-(3-bromo-benzylamino)-5-nitro-pyrimidin-4-ylamino]-methyl}-cyclohexylmethyl)-carbamic acid tert-butyl ester as a pale yellow solid, m/z 549.3 (M+H)⁺.

To a mixture of (trans-4-{[2-(3-bromo-benzylamino)-5-nitro-pyrimidin-4-ylamino]-methyl}-cyclohexylmethyl)-carbamic acid tert-butyl ester (50 mg, 0.091 mmol), (3-aminomethylphenyl)boronic acid HCl (26 mg, 0.137 mmol), tetrakis(triphenylphosphine)palladium (10 mg, 0.009 mmol), and sodium carbonate (38 mg, 0.360 mmol) was added dimethoxyethane (1.0 mL) and water (0.150 mL). The reaction mixture was sealed under N₂ and heated at 90° C. for 5 h. The reaction mixture was partitioned between ethyl acctate (20 mL) and water (5 mL). The organic phase was washed with brine, dried over Na₂SO₄, filtered and concentrated. The residue was purified by silica gel chromatography eluting with 0-80% 0.1:1:9 NH₄OH/MeOH/CH₂Cl₂ in CH₂Cl₂ to furnish 21 mg (40%) of [trans-4-({2-[(3′-aminomethyl-biphenyl-3-ylmethyl)-amino]-5-nitro-pyrimidin-4-ylamino}-methyl)-cyclohexylmethyl]-carbamic acid tert-butyl ester as a yellow oil, mm/z 576.4 (M+H)¹.

A solution of [trans-4-({2-[(3′-aminomethyl-biphenyl-3-ylmethyl)-amino]-5-nitro-pyrimidin-4-ylamino}-methyl)-cyclohexylmethyl]-carbamic acid tert-butyl ester (21 mg, 0.036 mmol) in dichloromethane (4.0 mL) was treated with 4 M HCl in dioxane (0.100 mL, 0.400 mmol). The reaction mixture was stirred at room temperature for 18 h and then concentrated. The crude product was purified silica gel chromatography eluting with 0-100% 0.1:1:9 NH₄OH/MeOH/CH₂Cl₂ in CH₂Cl₂ to give 16 mg (93%) of N²-{[3′-(aminomethyl)biphenyl-3-yl]methyl}-N⁴-{[trans-4-(aminomethyl)cyclohexyl]methyl}-5-nitropyrimidine-2,4-diamine, m/z 476.5 (M+H)⁺.

The compounds according to Examples 21-25 as presented in Table 1 may be prepared by a procedure analogous to that described above in Example 1. In some cases, trifluoroacetic acid was substituted for HCl/dioxane in the final boc deprotection step. In some instances, the crude product of the final deprotection step was isolated after an aqueous workup in which the excess acid was neutralized with a base such as satd NaHCO₃ solution, and the desired product was extracted with dichloromethane.

The compound according to Example 26 as presented in Table 1 may prepared by a procedure analogous to that described above in Example 1 and 21-25 by using (trans-4-aminomethyl-cyclohexyl)-carbamic acid tert-butyl ester as starting material.

The compound according to Example 27 as presented in Table 1 may prepared by a procedure analogous to that described above in Example 1 by using [4-({2-[(5-bromo-pyridin-3-ylmethyl)-amino]-5-nitro-pyrimidin-4-ylamino}-methyl)-cyclohexylmethyl]-carbamic acid tert-butyl ester as starting material.

### Example 2

### Synthesis of 3-({4-[(4-aminomethyl-cyclohexylmethyl)-amino]-5-nitro-pyrimidin-2-ylamino}-methyl)-N-(2-hydroxy-ethyl)-benzamide

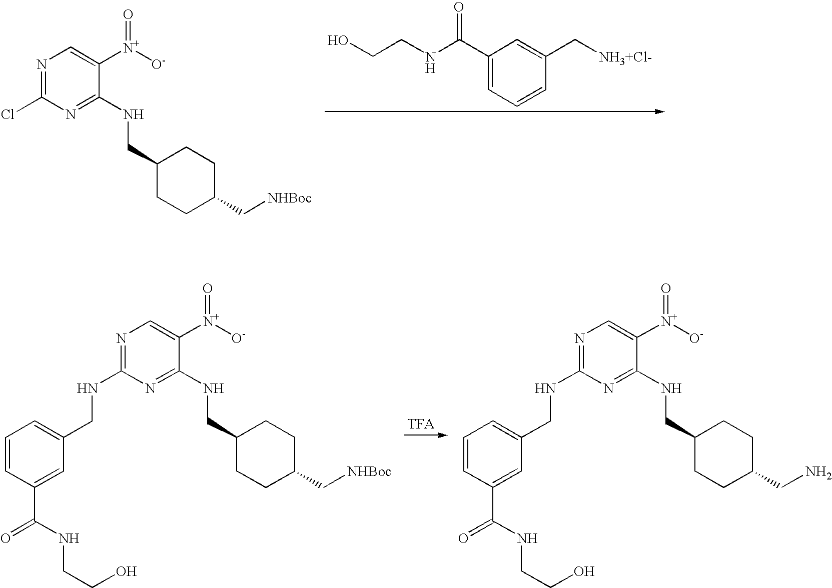

trans-4-[(2-Chloro-5-nitro-pyrimidin-4-ylamino)-methyl]-cyclohexylmethyl-carbamic acid tert-butyl ester (1.00 g, 2.50 mmol) was placed in a round bottomed flask with DMF (20 m/L) and Hunigs Base (2.0 mL). 3-Aminomethyl-N-(2-hydroxy-ethyl)-benzamide (0.56 g, 3.00 mmol) was added and the reaction was allowed to stir for 14 h at rt. The volatiles were concentrated off in vacuo to afford [4-({2-[3-(2-hydroxy-ethylcarbamoyl)-benzylamino]-5-nitro-pyrimidin-4-ylamino}-methyl)-cyclohexylmethyl]-carbamic acid tert-butyl ester.

[4-({2-[3-(2-Hydroxy-ethylcarbamoyl)-benzylamino]-5-nitro-pyrimidin-4-ylamino}-methyl)-cyclohexylmethyl]-carbamic acid tert-butyl ester was placed in a round bottomed flask with dichloromethane and cooled to 0° C. Trifluoroacetic acid (1.0 mL) was added and the reaction was allowed to stir for 16 h. The volatiles were removed in vacuo. The resulting crude material was purified by column chromatography employing 10% Methanol, 90% Dichloromethane, and 0.1% Ammonium hydroxide as eluent to afford 3-({4-[(4-aminomethyl-cyclohexylmethyl)-amino]-5-nitro-pyrimidin-2-ylamino}-methyl)-N-(2-hydroxy-ethyl)-benzamide, m/z 458.6 (M+H)⁺.

The compounds according to Examples 28-29 as presented in Table 1 may be prepared by a procedure analogous to that described above in Example 2.

### Synthesis of 3-aminomethyl-N-(2-hydroxy-ethyl)-benzamide

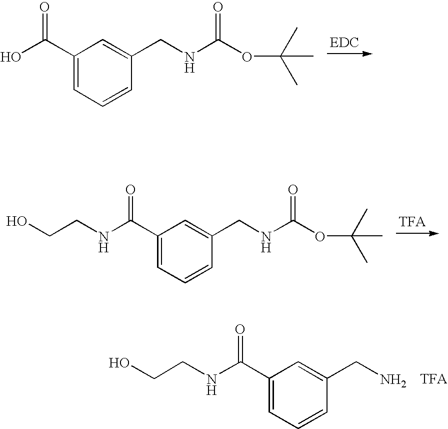

3-(tert-butoxycarbonylamino-methyl)-benzoic acid (0.30 g, 1.19 mmol) was placed in a round bottomed flask with dichloromethane (10 mL) and cooled to 0° C. EDC (0.25 g, 1.30 mmol) was added and the reaction was allowed to stir for 30 min at 0° C. Ethanolamine (0.09 g, 1.40 mmol) and DMAP (5 mg) were added and the reaction was allowed to warm to rt and stir for 14 h. The reaction was diluted with dichloromethane (20 mL) and washed with water (3×10 mL). The organics were dried, concentrated, and taken back up in dichloromethane (10 mL) and re-cooled to 0° C. Trifluoroacetic acid (1.0 mL) was added and the reaction was allowed to stir for 16 h. The volatiles were removed in vacuo and the resulting 3-aminomethyl-N-(2-hydroxy-ethyl)-benzamide was taken onward without further purification.

3-Aminomethyl-N-(2-morpholin-4-yl-ethyl)-benzamide and 3-aminomethyl-N-(2-dimethylamino-ethyl)-benzamide were prepared by a procedure analogous to that described above.

### Synthesis of 3-piperidin-1-yl-benzylamine

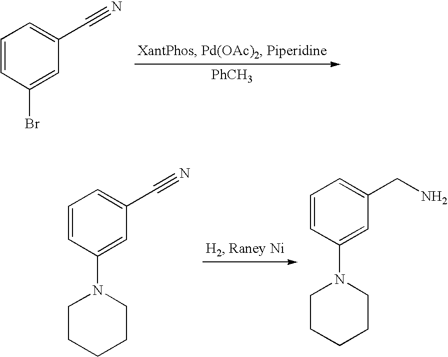

Palladium (II) acetate (12.3 mg, 0.055 mmol) and 4,5-bis(diphenylphosphino)-9,9-dimethyxanthene (47.7 mg, 0.082 mmol, Xantphos) were combined and the flask was evacuated and flushed three times with N₂. Degassed PhCH₃ (25 mL) was added and the solution was stirred for 5 min. 3-Bromobenzonitrile (1.0 g, 5.49 mmol) and piperidine (561 mg, 6.59 mmol) were added and the reaction was stirred for another 5 min. Cs₂CO₃ (2.15 g, 6.59 mmol) was added and the flasked was flushed with N₂ for 1 min then heated to 70° C. for 48 h. The volatiles were removed to afford 965 mg of 3-piperidin-1-yl-benzonitrile which was carried further without purification.

3-Piperidin-1-yl-benzonitrile (965 mg, 5.18 mmol) was dissolved in MeOH (125 mL). To this solution was added about 1.5 mL of Raney Nickel suspension in H₂O. The flask was evacuated and back-flushed with N₂. A balloon was filled with H₂ and the reaction flask was evacuated, filled with H₂, and maintained under atmospheric pressure. The reaction was stirred vigorously for 2 h, then filtered through a 2 cm thick pad of Celite under a stream of N₂. The volatiles were removed to afford 993 mg of 3-piperidin-1-yl-benzylamine which was carried further without purification.

3-pyrrolidin-1-yl-benzylamine and 3-azepan-1-yl-benzylamine were prepared by a procedure analogous to that described above.

### Example 3

### Synthesis of: N-(2-amino-ethyl)-3-({4-[(4-aminomethyl-cyclohexylmethyl)-amino]-5-nitro-pyrimidin-2-ylamino}-methyl)-benzamide

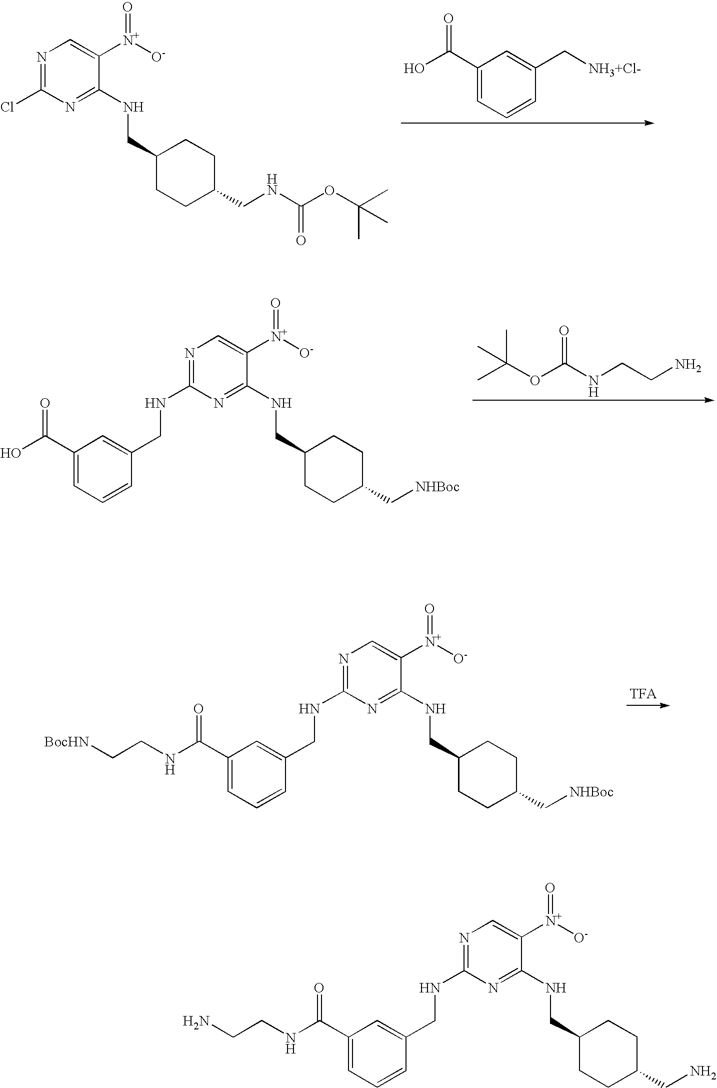

trans-4-[(2-chloro-5-nitro-pyrimidin-4-ylamino)-methyl]-cyclohexylmethyl}-carbamic acid tert-butyl ester (0.50 g, 1.25 mmol) was placed in a round bottomed flask with DMF (5 mL) and Hunigs Base (1.0 mL). 3-Aminomethyl-benzoic acid (0.30 g, 1.62 mmol) was added and the reaction was allowed to stir at rt for 16 h. The volatiles were removed and the resulting crude material was purified by column chromatography employing 5% Methanol, 95% Dichloromethane, and 0.1% Ammonium hydroxide as eluent to afford 3-[(4-{[4-(tert-butoxycarbonylamino-methyl)-cyclohexylmethyl]-amino}-5-nitro-pyrimidin-2-yl amino)-methyl]-benzoic acid.

3-[(4-{[4-(tert-butoxycarbonylamino-methyl)-cyclohexylmethyl]-amino}-5-nitro-pyrimidin-2-ylamino)-methyl]-benzoic acid (0.13 g, 0.24 mmol) was placed in a round bottomed flask with DMF (5 mL). TBTU (0.09 g, 0.27 mmol) was added and the reaction was allowed to stir for 20 min. (2-Amino-ethyl)-carbamic acid tert-butyl ester (0.05 g, 0.29 mmol) was added and the reaction was allowed to stir at rt for 14 h. The reaction was diluted with dichloromethane (20 mL) and washed with water (3×10 mL). The organics were separated, dried (MgSO₄) and concentrated. The resulting crude material was purified by column chromatography employing 5% Methanol, 95% Dichloromethane, and 0.1% Ammonium hydroxide as eluent to afford (2-{3-[(4-{[4-(tert-butoxycarbonylamino-methyl)-cyclohexylmethyl]-amino}-5-nitro-pyrimidin-2-ylamino)-methyl]-benzoylamino}-ethyl)-carbamic acid tert-butyl ester

(2-{3-[(4-{[4-(tert-butoxycarbonylamino-methyl)-cyclohexylmethyl]-amino}-5-nitro-pyrimidin-2-ylamino)-methyl]-benzoylamino}-ethyl)-carbamic acid tert-butyl ester (0.18 g, 0.27 mmol) was placed in a round bottomed flask with dichloromethane (5 mL) and cooled to 0° C. Trifluoroacetic acid (1.0 mL) was added and the reaction was allowed to stir for 16 h. The volatiles were removed in vacuo. The resulting crude material was purified by column chromatography employing 10% Methanol, 90% Dichloromethane, and 0.1% Ammonium hydroxide as eluent to afford N-(2-amino-ethyl)-3-({4-[(4-aminomethyl-cyclohexylmethyl)-amino]-5-nitro-pyrimidin-2-ylamino}-methyl)-benzamide, m/z 457.5 (M+H)⁺.

### Example 4

### N⁴-[(trans-4-aminocyclohexyl)methyl]-N²-{[3′-(aminomethyl)-2-methylbiphenyl-3-yl]methyl}-5-nitropyrimidine-2,4-diamine

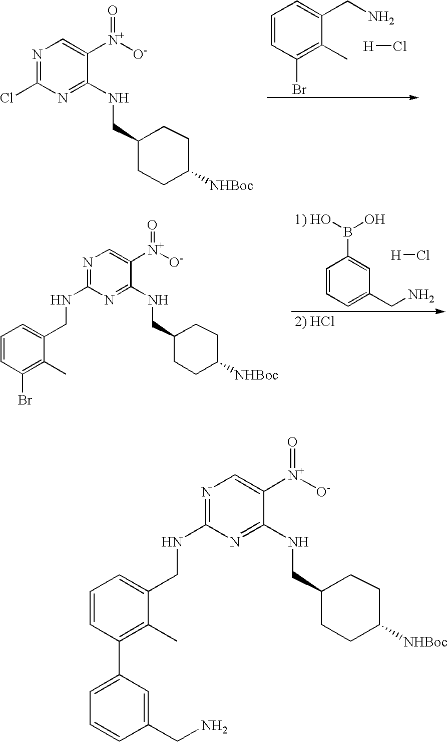

To a solution of 3-bromo-2-methyl-benzylamine hydrochloride (539 mg, 2.28 mmol) and diisopropylethylamine (0.72 mL, 4.15 mmol) in dichloromethane (25 mL) was added {trans-4-[(2-chloro-5-nitro-pyrimidin-4-ylamino)-methyl]-cyclohexyl}-carbamic acid tert-butyl ester (800 mg, 2.07 mmol). The r×n mixture was stirred at room temperature for 17 h, then concentrated, triturated with methanol, and filtered to provide (trans-4-{[2-(3-bromo-2-methyl-benzylamino)-5-nitro-pyrimidin-4-ylamino]-methyl}-cyclohexyl)-carbamic acid tert-butyl ester. The product was carried on without further purification.

To a mixture of (trans-4-{[2-(3-bromo-2-methyl-benzylamino)-5-nitro-pyrimidin-4-ylamino]-methyl}-cyclohexyl)-carbamic acid tert-butyl ester (200 mg, 0.36 mmol), (3-aminomethylphenyl)boronic acid HCl (107 mg, 0.55 mmol), tetrakis(triphenylphosphine)palladium (42 mg, 0.04 mmol), and sodium carbonate (150 mg, 1.42 mmol) was added dimethoxyethane (3.0 mL) and water (0.10 mL). The reaction mixture was sealed under N₂ and heated at 140° C. in the microwave for 2 h. The reaction mixture was partitioned between ethyl acctate (20 mL) and water (5 mL). The organic phase was washed with brine, dried over Na₂SO₄, filtered and concentrated. The residue was purified by silica gel chromatography eluting with 0-50% 0.1:1:9 NH₄OH/MeOH/CH₂Cl₂ in CH₂Cl₂ to furnish 147 mg of [trans-4-({2-[(3′-aminomethyl-2-methyl-biphenyl-3-ylmethyl)-amino]-5-nitro-pyrimidin-4-ylamino}-methyl)-cyclohexyl]-carbamic acid tert-butyl ester.

A solution of [trans-4-({2-[(3′-aminomethyl-2-methyl-biphenyl-3-ylmethyl)-amino]-5-nitro-pyrimidin-4-ylamino}-methyl)-cyclohexyl]-carbamic acid tert-butyl ester (147 mg, 0.26 mmol) in dichloromethane (10 mL) was treated with 4 M HCl in dioxane (0.640 mL, 2.56 mmol). The reaction mixture was stirred at room temperature for 1 h and then concentrated to afford 125 mg (89%) of N⁴-[(trans-4-aminocyclohexyl)methyl]-N²-{[3′-(aminomethyl)-2-methylbiphenyl-3-yl]methyl}-5-nitropyrimidine-2,4-diamine, m/z 474.5 (M−H)⁻.

{trans-4-[(2-chloro-5-nitro-pyrimidin-4-ylamino)-methyl]-cyclohexyl}-carbamic acid tert-butyl ester was prepared by a procedure analogous to that described in Example 1 using (trans-4-aminomethyl-cyclohexyl)-carbamic acid tert-butyl ester as starting material. The compounds according to Example 30 as presented in Table 1 may was prepared by a procedure analogous to that described above in Example 4 using 3-bromo-2-chloro-benzylamine as starting material.

The compounds according to Examples 31-34 as presented in Table 1 may prepared by a procedure analogous to that described above in Example 4 by using {trans-4-[(2-chloro-5-nitro-pyrimidin-4-ylamino)-methyl]-cyclohexylmethyl}-carbamic acid tert-butyl ester as starting material.

### Example 5

### Synthesis of N²-(3′-Aminomethyl-2-methyl-biphenyl-3-ylmethyl)-5-nitro-N⁴-piperidin-4-ylmethyl-pyrimidine-2,4-diamine

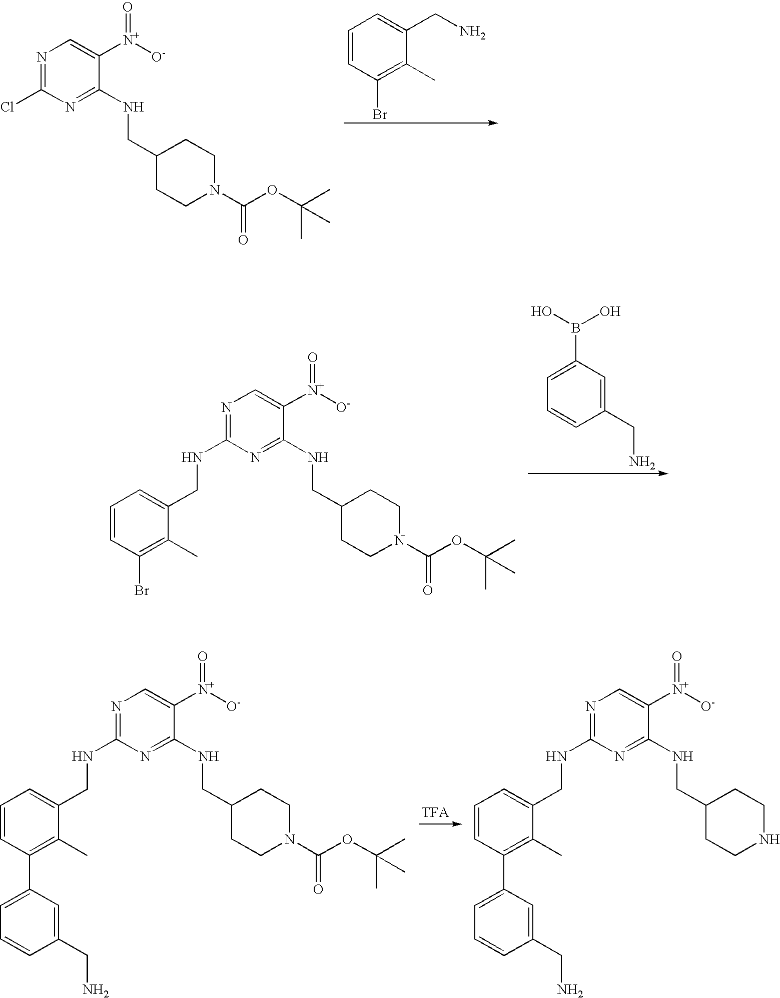

To a solution of 4-[(2-chloro-5-nitro-pyrimidin-4-ylamino)-methyl]-piperidine-1-carboxylic acid tert-butyl ester (786 mg, 2.11 mmol) in dichloromethane (1.2 mL) was added 3-bromo-2-methyl-benzylamine (550 mg, 2.32 mmol) and diisopropylethylamine (0.55 mL, 3.17 mmol) and the mixture was stirred at room temperature overnight. The solution was concentrated and then the compound was triturated with methanol. The product was filtered to afford 516 mg (46%) of 4-{[2-(3-bromo-2-methyl-benzylamino)-5-nitro-pyrimidin-4-ylamino]-methyl}-piperidine-1-carboxylic acid tert-butyl ester as a pale yellow solid, m/z 536.4 (M+H)⁺.

To a mixture of 4-{[2-(3-bromo-2-methyl-benzylamino)-5-nitro-pyrimidin-4-ylamino]-methyl}-piperidine-1-carboxylic acid tert-butyl ester (150 mg, 0.280 mmol), (3-aminomethylphenyl)boronic acid HCl (79 mg, 0.420 mmol), tetrakis(triphenylphosphine)palladium (32 mg, 0.028 mmol), and potassium carbonate (155 mg, 1.12 mmol) was added dimethoxyethane (2.0 mL) and water (0.2 mL). The reaction mixture was sealed under N₂ and heated at 190° C. in the microwave for 5 minutes. The reaction mixture was diluted with saturated NaHCO₃ and dichloromethane. The phases were separated and the organic phase was dried over Na₂SO₄. The solution was concentrated and the material was purified by silica gel chromatography eluting with 10% MeOH in CH₂Cl₂ to furnish 86 mg (55%) of 4-({2-[(3′-aminomethyl-2-methyl-biphenyl-3-ylmethyl)-amino]-5-nitro-pyrimidin-4-ylamino}-methyl)-piperidine-1-carboxylic acid tert-butyl ester, m/z 562.7 (M+H)⁺

To a solution of 4-({2-[(3′-aminomethyl-2-methyl-biphenyl-3-ylmethyl)-amino]-5-nitro-pyrimidin-4-ylamino}-methyl)-piperidine-1-carboxylic acid tert-butyl ester (86 mg, 0.153 mmol) in dichloromethane (0.86 mL) was added trifluoroacetic acid (0.43 mL). The mixture was stirred at room temperature for 4 hours and was concentrated to afford 140 mg (100%) of N²-(3′-aminomethyl-2-methyl-biphenyl-3-ylmethyl)-5-nitro-N⁴-piperidin-4-ylmethyl-pyrimidine-2,4-diamine, m/z 462.6 (M+H)⁺.

### Example 6

### Synthesis of [trans-4-({[2-({[3′-(aminomethyl)biphenyl-3-yl]methyl}amino)-5-nitropyrimidin-4-yl]amino}methyl)cyclohexyl]methanol

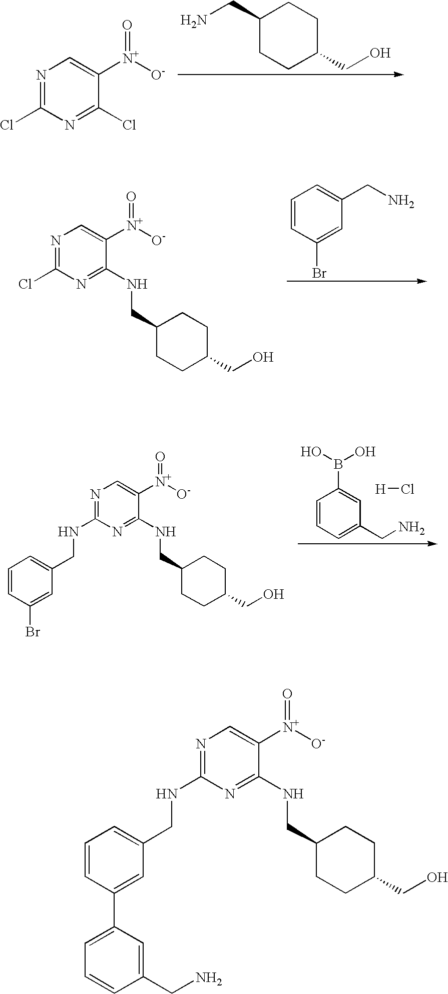

To a solution of trans-(4-aminomethyl-cyclohexyl)-methanolamine trifluoroacetic acid salt (1.58 g, 6.16 mmol) and diisopropylethylamine (4.50 mL, 25.8 mmol) in 35 mL of dichloromethane was added 2,4-dichloro-5-nitropyrimidine (1.19 g, 6.16 mmol). The reaction mixture was stirred at room temperature for 15 h, then diluted with dichloromethane (15 mL) and washed with 1 M HCl solution (35 mL). The organic phase was washed with satd NaHCO₃ solution (30 mL) and brine (40 mL), dried over Na₂SO₄, filtered and concentrated. The crude product was purified by silica gel chromatography eluting with 25-50% ethyl acetate in hexanes to afford 771 mg (42%) of trans-{4-[(2-chloro-5-nitro-pyrimidin-4-ylamino)-methyl]-cyclohexyl}-methanol as a yellow solid, m/z 301.5 (M+H)⁺.

To a solution of 3-bromo-benzylamine (190 mg, 1.02 mmol) and diisopropylethylamine (0.175 mL, 1.01 mmol) in dichloromethane (5 mL) was added trans-{4-[(2-chloro-5-nitro-pyrimidin-4-ylamino)-methyl]-cyclohexyl}-methanol (215 mg, 0.715 mmol). The r×n mixture was stirred at room temperature for 15 h, then partitioned between dichloromethane (50 mL) and 1M HCl solution (10 mL). The organic phase was washed with satd NaHCO₃ solution (10 mL) and brine (15 mL), dried over Na₂SO₄, filtered and concentrated. The crude product was purified by silica gel chromatography eluting with 0-4% MeOH in CH₂Cl₂ to afford 170 mg (53%) of (trans-4-{[2-(3-bromo-benzylamino)-5-nitro-pyrimidin-4-ylamino]-methyl}-cyclohexyl)-methanol as a yellow solid, m/z 450.5 (M+H)⁺.

To a mixture of (trans-4-{[2-(3-bromo-benzylamino)-5-nitro-pyrimidin-4-ylamino]-methyl}-cyclohexyl)-methanol (74 mg, 0.164 mmol), 3-(aminomethylphenyl)boronic acid, HCl salt (46 mg, 0.246 mmol), tetrakis(triphenylphosphine)palladium (18 mg, 0.016 mmol), and sodium carbonate (70 mg, 0.656 mmol) was added dimethoxyethane (1.5 mL) and water (0.200 mL). The reaction mixture was sealed under N₂ and heated at 90° C. for 9 h. The reaction mixture was partitioned between dichloromethane (30 mL) and 5% NaCl solution (8 mL). The organic phase was washed with brine, dried over Na₂SO₄, filtered and concentrated. The residue was purified by silica gel chromatography cluting with 0-80% 0.1:1:9 NH₄OH/MeOF/CH₂Cl₂ in CH₂Cl₂ to furnish 31 mg (37%) of [trans-4-({2-[(3′-aminomethyl-biphenyl-3-ylmethyl)-amino]-5-nitro-pyrimidin-4-ylamino}-methyl)-cyclohexyl]-methanol as a yellow foam, m/z 477.5 (M+H)⁺.

### Example 7

### Synthesis of {trans-4-[(2-{2-[3-(4-aminomethyl-piperidin-1-yl)-phenyl]-ethyl}-5-nitro-pyrimidin-4-ylamino)-methyl]-cyclohexyl}-methanol

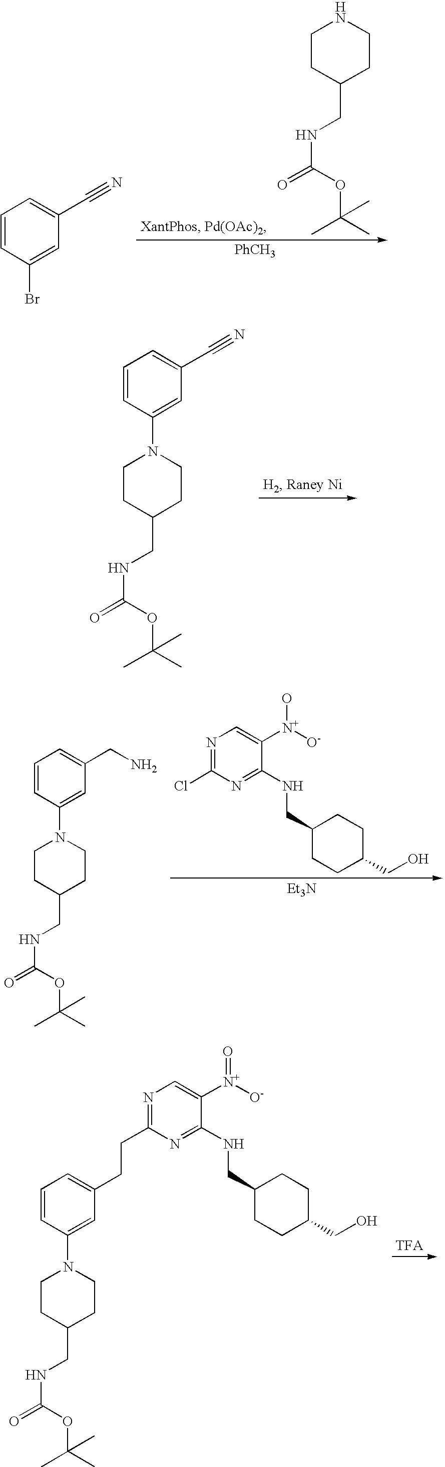

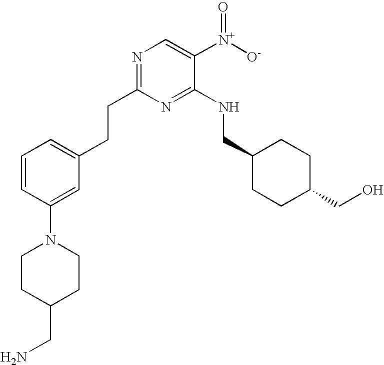

Palladium (II) acetate (37 mg, 0.165 mmol) and 4,5-bis(diphenylphosphino)-9,9-dimethyxanthene (143 mg, 0.247 mmol, Xantphos) were combined and the flask was evacuated and flushed three times with N₂. Degassed PhCH₃ (25 mL) was added and the solution was stirred for 5 min. 3-Bromobenzonitrile (1.0 g, 5.49 mmol) and Boc-4-(aminomethyl)-piperidine (1.4 g, 6.59 mmol) were added and the reaction was stirred for another 5 min. Cs₂CO₃ (2.15 g, 6.59 mmol) was added and the flask was flushed with N₂ for 1 min then heated to 90° C. for 24 h. The volatiles were removed and the crude residue was purified via flash chromatography (SiO₂, 25-60% EA-Hexanes) to afford 1.27 g (73%) of [1-(3-cyano-phenyl)-piperidin-4-ylmethyl]-carbamic acid tert-butyl ester. [1-(3-Cyano-phenyl)-piperidin-4-ylmethyl]-carbamic acid tert-butyl ester (1.27 g, 4.03 mmol) was dissolved in MeOH (300 mL). To this solution was added about 1.5 mL of Raney Nickel suspension in H₂O. The flask was evacuated and backflushed with N₂, evacuated, filled with H₂, and maintained under balloon pressure. The reaction was stirred vigorously for 24 h then filtered through a 2 cm thick pad of Celite under a stream of N₂. The volatiles were removed and the crude residue was purified via flash chromatography (SiO₂, 10-50% ((2:18:80-NH₄OH:MeOH:CH₂Cl₂):CH₂Cl₂) to afford 455 mg (35%) of [1-(3-aminomethyl-phenyl)-piperidin-4-ylmethyl]-carbamic acid tert-butyl ester.

Trans-{4-[(2-chloro-5-nitro pyrimidin-4-ylamino)-methyl]-cyclohexyl}-methanol (100 mg, 0.33 mmol) was dissolved in CH₂Cl₂ (10 mL) followed by Et₃N (0.116 mL, 0.83 mmol). [1-(3-Aminomethyl-phenyl)-piperidin-4-ylmethyl]-carbamic acid tert-butyl ester (117 mg, 0.37 mmol) was dissolved in DMA:EtOH (2 mL, 1:1) and added to the reaction. The reaction was stirred at room temperature for 18 h, the volatiles removed and the crude purified via flash chromatography (SiO₂, 5% MeOH—CH₂Cl₂) to afford {1-[3- (2-{4-[(trans-4-hydroxymethyl-cyclohexylmethyl)-amino]-5-nitro-pyrimidin-2-yl}-ethyl)-phenyl]-piperidin-4-ylmethyl}-carbamic acid tert-butyl ester which was redissolved in CH₂Cl₂ (10 mL). TFA (10 mL) was added and the reaction was stirred for 3 h. The crude was purified via flash chromatography (SiO₂, 50-100% ((NH₄OH:MeOH:CH₂Cl₂-(2:18:80)CH₂Cl₂) to afford 136 mg of {trans-4-[(2-{2-[3-(4-aminomethyl-piperidin-1-yl)-phenyl]-ethyl}-5-nitro-pyrimidin-4-ylamino)-methyl]-cyclohexyl}-methanol (85%), m/z 484.1 (M+H)⁺.

The compounds according to Examples 35-37 as presented in Table 1 may be prepared by a procedure analogous to that described above in Example 7 and by utilizing intermediates described herein.

### Example 8

### Synthesis of N-[3-({[4-({[trans-4-(hydroxymethyl)cyclohexyl]methyl}amino)-5-nitropyrimidin-2-yl]amino}methyl)phenyl]glycinamide

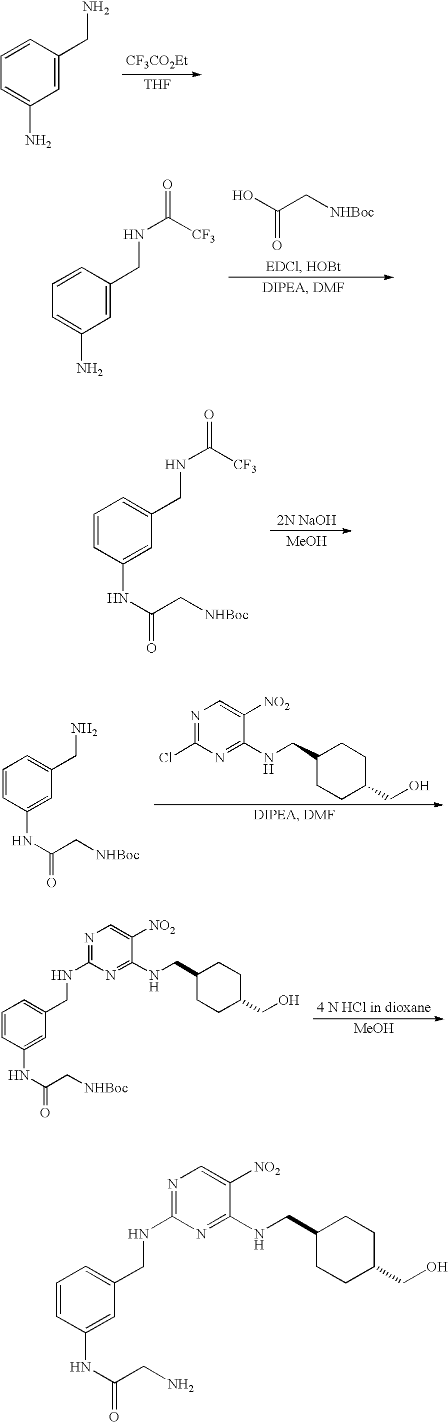

To a solution of 3-aminobenzylamine (3.76 g, 30.10 mmol) in THF (60 mL) was added ethyl trifluoroacetate (4.07 mL, 34.10 mmol) via syringe. The reaction mixture was stirred at room temperature under N₂ overnight before concentrating in vacuo to afford N-(3-amino-benzyl)-2,2,2-trifluoro-acetamide (6.64 g, 98%) as an orange oil.

To a solution of N-(3-amino-benzyl)-2,2,2-trifluoro-acetamide (1.50 g, 6.86 mmol) in DMF (15 mL) was added successively N,N-diisopropylethylamine (2.96 mL, 17.05 mmol), N-Boc-glycine (1.00 g, 5.71 mmol), HOBt (1.08 g, 8.00 mmol) and EDCI (1.52 g, 8.00 mmol). The reaction was stirred under N₂ at room temperature overnight before addition of water. The mixture was extracted with twice EtOAc and the combined extracts washed with water and brine. Concentration in vacuo afforded an oil which was purified by column chromatography using an ISCO combi-flash cartridge (silica gel, hexane/EtOAc) to afford ({3-[(2,2,2-trifluoro-acetylamino)-methyl]-phenylcarbamoyl}-methyl)-carbamic acid tert-butyl ester (1.75 g, 82%) as an off-white solid.

To a solution of ({3-[(2,2,2-trifluoro-acetylamino)-methyl]-phenylcarbamoyl}-methyl)-carbamic acid tert-butyl ester (1.34 g, 3.58 mmol) in MeOH (10 mL) was added 2 N NaOH (1.3 mL) and the solution stirred at room temperature. Further portions of 2 N NaOH were added after 30 min (1.0 mL) and 1 h (3.0 mL). After an additional 1 h the reaction mixture was concentrated in vacuo and the aqueous residue extracted with EtOAc. The organic extract was concentrated in vacuo to afford [(3-aminomethyl-phenylcarbamoyl)-methyl]-carbamic acid tert-butyl ester (0.81 g, 80%) as a viscous oil.

To a solution of trans-{4-[(2-chloro-5-nitro-pyrimidin-4-ylamino)-methyl]-cyclohexyl}-methanol (300 mg, 1.00 mmol) in DMF (3 mL) was added a solution of [(3-aminomethyl-phenylcarbamoyl)-methyl]-carbamic acid tert-butyl ester (279 mg, 1.00 mmol) and N,N-diisopropylethylamine (0.21 mL, 1.20 mmol) in DMF (3 mL). The reaction mixture was stirred under N₂ overnight at room temperature before partitioning between EtOAc and water. The organic layer was separated and washed with water and sat. NaHCO₃ before concentrating in vacuo. The residue was purified by column chromatography using an ISCO combi-flash cartridge (silica gel, hexane/EtOAc) to afford {trans-[3-({4-[(4-hydroxymethyl-cyclohexylmethyl)-amino]-5-nitro-pyrimidin-2-ylamino}-methyl)-phenylcarbamoyl]-methyl}-carbamic acid tert-butyl ester (422 mg, 78%) as an orange oil, m/z 544 (M+H)⁺.

To a solution of the boc amine (422 mg, 0.78 mmol) in MeOH (1 mL) was added 4 N HCl in dioxane (2 mL). After 30 min the reaction mixture was concentrated in vacuo before it was dissolved in a small amount of MeOH and treated with excess concd. NH₄OH. The solid which precipitated was filtered and washed with water. This material was purified by semi-preparative HPLC(C₁₈ column using 10-90% CH₃CN/H₂O gradient). Pure product fractions were concentrated in vacuo to afford the bis-TFA salt of N-[3-({[4-({[trans-4-(hydroxymethyl)cyclohexyl]methyl}amino)-5-nitropyrimidin-2-yl]amino}methyl)phenyl]glycinamide (114 mg, 21%) as a yellow solid, m/z 444 (M+H)⁺.

The compounds according to Example 38 as presented in Table 1 may prepared by a procedure analogous to that described above in Example 8 by utilizing N-Boc-β-alanine as starting material in step 2:

### Example 9

### Synthesis of [trans-4-({[2-({[3′-(aminomethyl)-2-methylbiphenyl-3-yl]methyl}amino)-5-nitropyrimidin-4-yl]amino}methyl)cyclohexyl]methanol

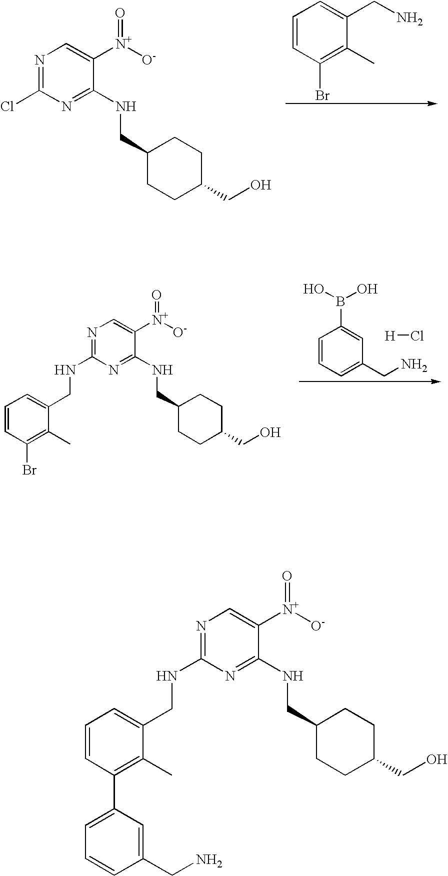

To a solution of 3-bromo-2-methyl-benzylamine (134 mg, 0.670 mmol) and diisopropylethylamine (0.118 mL, 0.674 mmol) in dichloromethane (4 mL) was added trans-{4-[(2-chloro-5-nitro-pyrimidin-4-ylamino)-methyl]-cyclohexyl}-methanol (137 mg, 0.456 mmol). The r×n mixture was stirred at room temperature for 18 h, then partitioned between dichloromethane (40 mL) and 5% citric acid solution (10 mL). The organic phase was washed with satd NaHCO₃ solution (10 mL) and brine (15 mL), dried over Na₂SO₄, filtered and concentrated. The crude product was purified by silica gel chromatography eluting with 0-4% MeOH in CH₂Cl₂ to afford 196 mg (93%) of (trans-4-{[2-(3-bromo-2-methyl-benzylamino)-5-nitro-pyrimidin-4-ylamino]-methyl}-cyclohexyl)-methanol as a pale yellow solid, m/z 464.3 (M+H)⁺.

To a mixture of (trans-4-{[2-(3-bromo-2-methyl-benzylamino)-5-nitro-pyrimidin-4-ylamino]-methyl}-cyclohexyl)-methanol (161 mg, 0.347 mmol), 3-(aminomethylphenyl)boronic acid, HCl salt (114 mg, 0.607 mmol), tetrakis(triphenylphosphine)palladium (61 mg, 0.052 mmol), and sodium carbonate (147 mg, 1.39 mmol) was added dimethoxyethane (3 mL) and water (0.4 mL). The reaction mixture was sealed under N₂ and heated at 90° C. for 13 h. The reaction mixture was partitioned between dichloromethane (40 mL) and 5% NaCl solution (10 mL). The organic phase was washed with brine, dried over Na₂SO₄, filtered and concentrated. The residue was purified by silica gel chromatography eluting with 0-75% 0.1:1:9 NH₄OH/MeOH/CH₂Cl₂ in CH₂Cl₂ to furnish 60 mg (30%) of [trans-4-({[2-({[3′-(aminomethyl)-2-methylbiphenyl-3-yl]methyl}amino)-5-nitropyrimidin-4-yl]amino}methyl)cyclohexyl]methanol, m/z 491.6 (M+H)⁺.

The compounds according to Examples 39-43 as presented in Table 1 may be prepared by a procedure analogous to that described above in Example 9.

The compound according to Example 44 as presented in Table 1 may be prepared by a procedure analogous to that described above in Example 9 by using 3-bromo-2-fluoro-benzylamine as starting material.

The compound according to Example 45 as presented in Table 1 may be prepared by a procedure analogous to that described above in Example 9 by using 3-bromo-2-trifluoromethoxy-benzylamine as starting material.

The compound according to Example 46 as presented in Table 1 may be prepared by a procedure analogous to that described above in Example 9 by using 3-bromo-2-chloro-benzylamine as starting material.

### Synthesis of 2-[3-(4,4,5,5-Tetramethyl-[1,3,2]dioxaborolan-2-yl)-phenyl]-ethylamine

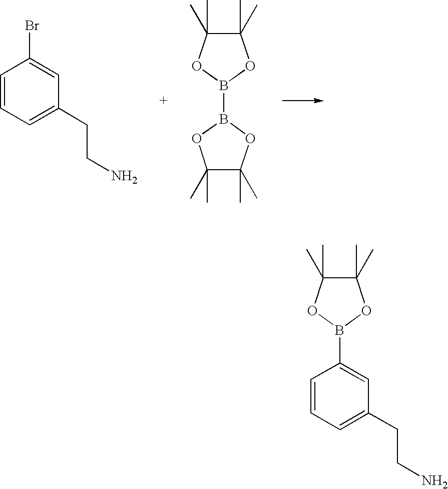

To a solution of 2-(3-bromo-phenyl)-ethylamine (500 mg, 2.5 mmol) and 4,4,5,5,4′,4′,5′,5′-octamethyl-[2,2′]bi[[1,3,2]dioxaborolanyl] (1.9 g, 7.5 mmol) in N,N-dimethylformamide (5.0 mL) was added potassium acctate (1.23 g, 12.5 mmol) and [1,1′-bis(diphenylphosphino)ferrocene]dichloropalladium(II) (185 mg, 0.250 mmol) and the solution was heated at 80° C. overnight. The solution was filtered through a plug of silica, washed with dichloromethane and the solvent was concentrated to afford the crude 2-[3-(4,4,5,5-tetramethyl-[1,3,2]dioxaborolan-2-yl)-phenyl]-ethylamine. The crude material was carried on without further purification.

### Example 10

### Synthesis of [trans-4-({2-[3-(3-amino-prop-1-ynyl)-2-methyl-benzylamino]-5-nitro-pyrimidin-4-ylamino}-methyl)-cyclohexyl]-methanol

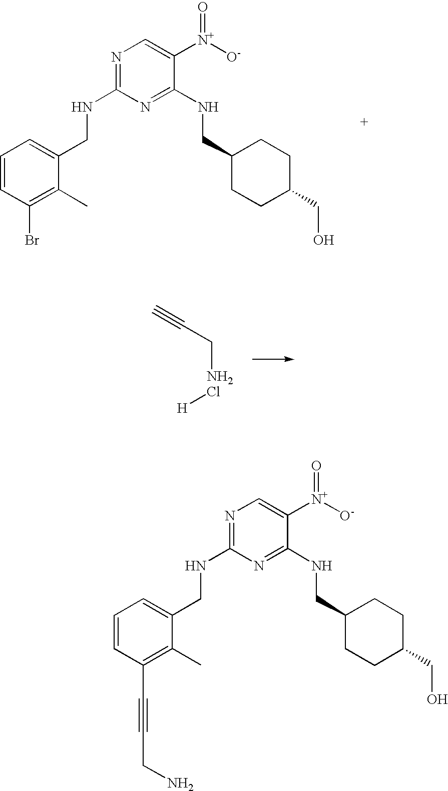

To a solution of (trans-4-{[2-(3-bromo-2-methyl-benzylamino)-5-nitro-pyrimidin-4-ylamino]-methyl}-cyclohexyl)-methanol (100 mg, 0.215 mmol) and prop-2-ynylamine hydrochloride (39.4 mg, 0.431 mmol) in triethylamine (300 μL, 2.15 mmol) and N,N-dimethylformamide (1.0 mL) was added copper (I) iodide (6.8 mg, 0.022 mmol) and bis(triphenylphosphine)palladium(II) chloride (9.7 mg, 0.013 mmol), and the solution was heated at 80° C. overnight. The solution was diluted with saturated NaHCO₃ and extracted with dichloromethane. The combined organics were dried over Na₂SO₄ and concentrated. The material was purified via silica gel chromatography using from 0-10% methanol in dichloromethane to afford 2.3 mg (2.4%) of [4-({2-[3-(3-amino-prop-1-ynyl)-2-methyl-benzylamino]-5-nitro-pyrimidin-4-ylamino}-methyl)-cyclohexyl]-methanol, m/z 439.5 (M+H)⁺.

### Example 11

### Synthesis of [trans-4-({[2-({[2′-(aminomethyl)-2-methylbiphenyl-3-yl]methyl}amino)-5-nitropyrimidin-4-yl]amino}methyl)cyclohexyl]methanol

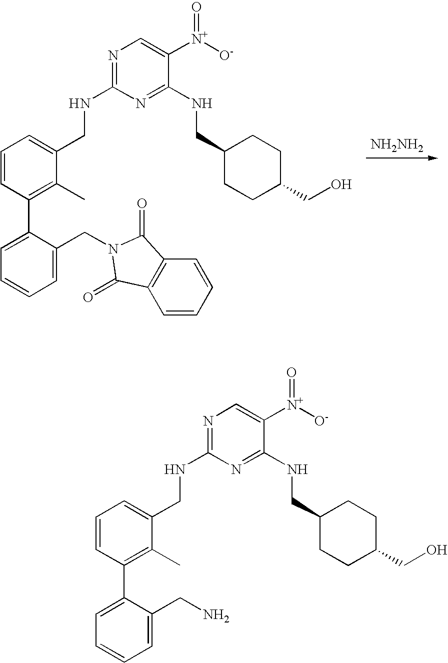

To a solution of 2-[3′-({4-[(4-hydroxymethyl-cyclohexylmethyl)-amino]-5-nitro-pyrimidin-2-ylamino}-methyl)-biphenyl-2-ylmethyl]-isoindole-1,3-dione (35 mg, 0.060 mmol) in 3 mL of EtOH—CH₂Cl₂ (2:1) was added hydrazine monohydrate (5 μL, 0.080 mmol). The reaction mixture was stirred at room temperature for 8 h. The reaction mixture was filtered and the filtrate was concentrated in vacuo to give a residue. The residue was purified using preparative silica gel chromatography (2% MeOH/CH₂Cl₂, 0.2% NH₄OH) to afford 15 mg (54%) of [trans-4-({[2-({[2′-(aminomethyl)-2-methylbiphenyl-3-yl]methyl}amino)-5-nitropyrimidin-4 yl]amino}methyl)cyclohexyl]methanol as a pale yellow solid, m/z 491.6 (M+H)⁺.

The starting material 2-[3′-({4-[(4-hydroxymethyl-cyclohexylmethyl)-amino]-5-nitro-pyrimidin-2-ylamino}-methyl)-biphenyl-2-ylmethyl]-isoindole-1,3-dione intermediate was prepared by a procedure analogous to that described above in Example 9.

### Example 12

### Synthesis of 3′-({[4-({[trans-4-(hydroxymethyl)cyclohexyl]-methyl}amino)-5-nitropyrimidin-2-yl]amino}methyl)-2′-methylbiphenyl-3-carbaldehyde

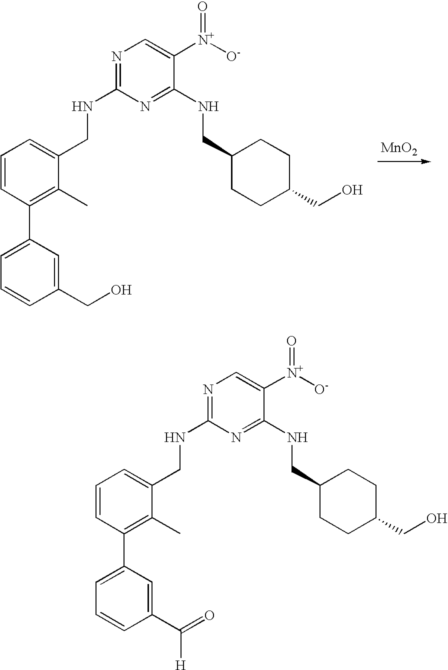

To a solution of [3′-({[4-({[trans-4-(hydroxymethyl)cyclohexyl]methyl}amino)-5-nitropyrimidin-2-yl]amino}methyl)-2′-methylbiphenyl-3-yl]methanol (0.190 g, 0.390 mmol) in THF (20 mL) was added MnO₂ (0.340 g, 3.91 mmol). The reaction mixture was stirred at room temperature for 1 h, then filtered through Celite and washed with ethyl acetate (15 mL). The solvent was removed using reduced pressure to afford a light-yellow foam. The crude product was purified by silica gel chromatography eluting with 0-25% EtOAc in Hexane to afford 130 mg (69%) of 3′-({[4-({[trans-4-(hydroxymethyl)cyclohexyl]-methyl}amino)-5-nitropyrimidin-2-yl]amino}methyl)-2′-methylbiphenyl-3-carbaldehyde as pale yellow foam, m/z 490.4 (+H)⁺.

### Example 13

### Synthesis of {trans-4-[({2-[({3′-[(dimethylamino)methyl]-2-methylbiphenyl-3-yl}methyl)amino]-5-nitropyrimidin-4-yl}amino)methyl]cyclohexyl}-methanol

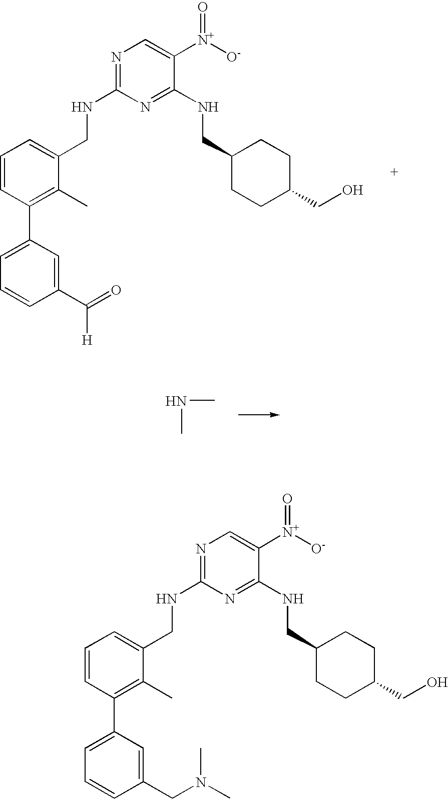

A round bottom flask equipped with a stir bar was charged with dimethylamine (0.300 mL), 3′-({[4-({[trans-4-(hydroxymethyl)cyclohexyl]-methyl}amino)-5-nitopyrimidin-2-yl]amino}methyl)-2′-methylbiphenyl-3-carbaldehyde (30 mg, 0.060 mmol), acetic acid (2 drops) and anhydrous sodium sulfate (10 mg). The reaction mixture was stirred under inert atmosphere at room temperature for 1 h, and then sodium triacetoxy-borohydride (28 mg, 0.130 mmol) was added. The mixture was stirred at room temperature for another 1 h. The reaction mixture was diluted with EtOAc (15 mL), quenched with satd aqueous Na₂CO₃ solution until pH=9. Combined organics were washed with brine and dried over MgSO₄, filtered and the solvent was removed in vacuo. The residue was purified using preparative silica gel chromatography (4% MeOH/CH₂Cl₂, 0.2% NH₄OH) to afford 25 mg (79%) of {trans-4-[({2-[({3′-[(dimethylamino)methyl]-2-methylbiphenyl-3-yl}methyl)amino]-5-nitropyrimidin-4-yl}amino)methyl]cyclohexyl}-methanol as pale yellow solid, m/z 519.5 (M+H)⁺.

The compounds according to Examples 47-50 as presented in Table 1 may be prepared by a procedure analogous to that described above in Example 13.

### Example 14

### Synthesis of 2-({[3′-({[4-({[trans-4-(hydroxymethyl)cyclohexyl]methyl}-amino)-5-nitropyrimidin-2-yl]amino}methyl)-2′-methylbiphenyl-3-yl]methyl}amino)-ethanol

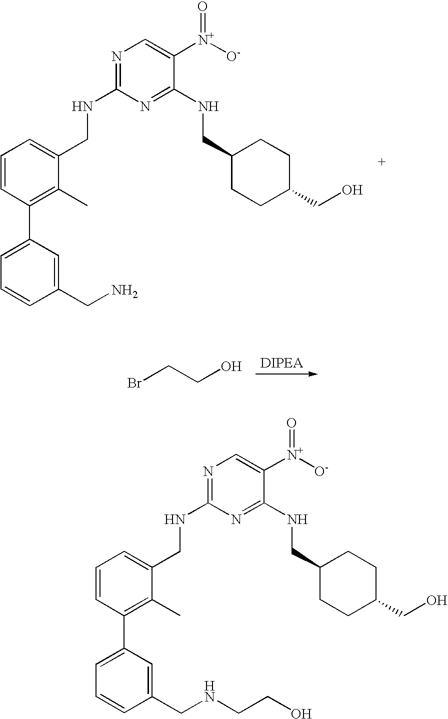

To a solution of [trans-4-({[2-({[3′-(aminomethyl)-2-methylbiphenyl-3-yl]methyl}amino)-5-nitropyrimidin-4-yl]amino}methyl)cyclohexyl]methanol (38 mg, 0.080 mmol) in DMF (2 mL) was added 2-bromo-ethanol (8 μL, 0.080 mmol) followed by diisopropylethylamine (14 μL, 0.080 mmol). The reaction mixture was stirred at room temperature for 8 h. The reaction mixture was diluted with EtOAc (10 mL) and washed with water (5 mL). The organic layers were separated, combined, dried over Na₂SO₄. After filtration, the filtrate was concentrated in vacuo. The residue was purified using preparative silica gel chromatography (5% MeOH/CH₂Cl₂) to afford 10 mg (24%) of 2-({[3′-({[4-({[trans-4-(hydroxymethyl)cyclohexyl]methyl}-amino)-5-nitropyrimidin-2-yl]amino}methyl)-2′-methylbiphenyl-3-yl]methyl}amino)-ethanol as pale-yellow solid, m/z 535.4 (M+H)⁺.

The compounds according to Examples 51-52 as presented in Table 1 may be prepared by a procedure analogous to that described in above Example 14.

### Example 15

### Synthesis of [trans-4-({[2-({3-[5-(aminomethyl)pyridin-3-yl]-2-methylbenzyl}amino)-5-nitropyrimidin-4-yl]amino}methyl)cyclohexyl]methanol

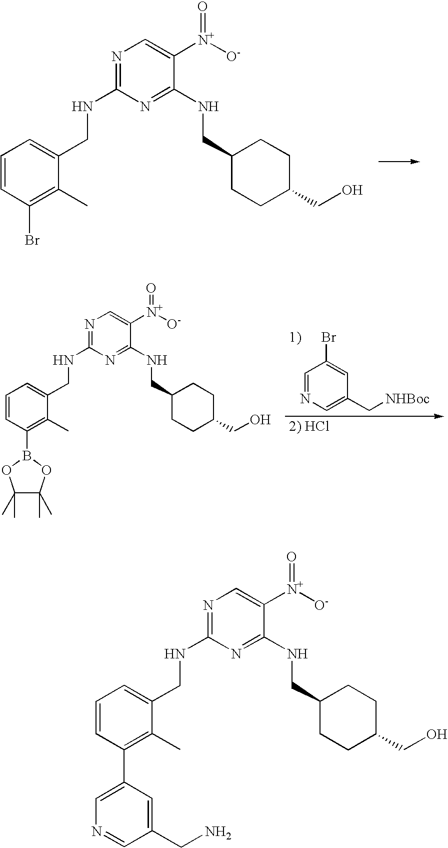

A mixture of (4-{[2-(3-bromo-2-methyl-benzylamino)-5-nitro-pyrimidin-4-ylamino]-methyl}-cyclohexyl)-methanol (131 mg, 0.28 mmol), bis(pinacolato)diboron (215 mg, 0.85 mmol), potassium acetate (138 mg, 1.41 mmol), and PdCl₂(dppf)₂.CH₂Cl₂ (23 mg, 0.03 mmol) in DMF (2.0 mL) was degassed with N₂. The reaction mixture was heated in a rescalable tube at 100° C. in the microwave for 1 h, then filtered through Celite, washing the filter cake with ethyl acetate. The filtrates were washed with 5% NaCl solution (3×15 mL) and brine (15 mL), dried over Na₂SO₄, filtered and concentrated. The residue was purified by silica gel chromatography eluting with 0-4% MeOH in CH₂Cl₂ to furnish 172 mg of [4-({2-[2-methyl-3-(4,4,5,5-tetramethyl-1,3,2-dioxaborolan-2-yl)-benzylamino]-5-nitro-pyrimidin-4-ylamino}-methyl)-cyclohexyl]-methanol as a yellow oil that was carried on without further purification.

To a mixture of the boronic ester (144 mg, 0.28 mmol), pyridyl bromide (121 mg, 0.42 mmol), tetrakis(triphenylphosphine)palladium (49 mg, 0.04 mmol), and potassium carbonate (117 mg, 0.85 mmol) was added dimethoxyethane (2.5 mL) and water (0.5 mL). The reaction mixture was sealed under N₂ and heated at 120° C. in the microwave for 2 h. The reaction mixture was filtered through Celite, washing the filter cake with ethyl acetate. The filtrates were washed with water and brine (20 mL each), dried over Na₂SO₄, filtered and concentrated. The residue was purified by silica gel chromatography eluting with 0-8% MeOH in CH₂Cl₂ to give 59 mg (35%) of {5-[3-({4-[(4-hydroxymethyl-cyclohexylmethyl)-amino]-5-nitro-pyrimidin-2-ylamino}-methyl)-2-methyl-phenyl]-pyridin-3-ylmethyl}-carbamic acid tert-butyl ester as an oil, m/z 592.2 (M+H)⁺.

A solution of {5-[3-({4-[(4-hydroxymethyl-cyclohexylmethyl)-amino]-5-nitro-pyrimidin-2-ylamino}-methyl)-2-methyl-phenyl]-pyridin-3-ylmethyl}-carbamic acid tert-butyl ester (58 mg, 0.10 mmol) in dichloromethane (2.0 mL) was treated with 4 M HCl in dioxane (0.125 mL, 0.49 mmol). The reaction mixture was stirred at room temperature for 3 h and then filtered, washing the solid with dichloromethane. The gummy solid was concentrated and then recrystallized from hot EtOH (10 mL) and dried in a vacuum oven (50° C.) to yield 26 mg (47%) of [trans-4-({[2-({3-[5-(aminomethyl)pyridin-3-yl]-2-methylbenzyl}amino)-5-nitropyrimidin-4-yl]amino}methyl)cyclohexyl]methanol as an off-white solid, isolated as the di-HCl salt, m/z 492.1 (M+H)⁺.

The compound according to Example 53 as presented in Table 1 may prepared by a procedure analogous to that described above in Example 15 by using (4-{[2-(3-bromo-2-chloro-benzylamino)-5-nitro-pyrimidin-4-ylamino]-methyl}-cyclohexyl)-methanol as starting material.

The compound according to Example 54 as presented in Table 1 may be prepared by a procedure analogous to that described above in Example 15 by using (4-{[2-(3-bromo-2-trifluoromethoxy-benzylamino)-5-nitro-pyrimidin-4-ylamino]-methyl}-cyclohexyl)-methanol as starting material.

### Synthesis of 3-bromo-2-methyl-benzylamine

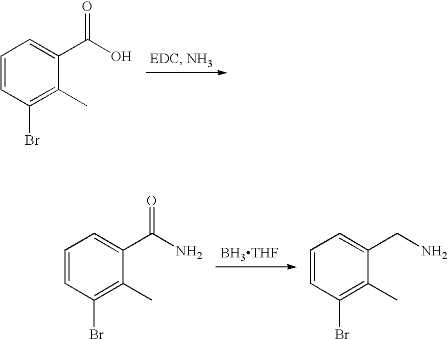

A solution of 3-bromo-2-methyl-benzoic acid (1.00 g, 4.65 mmol) and 1-hydroxybenzotriazole hydrate (637 mg, 4.71 mmol) in 1:1 dichloromethane/DMF (10 mL) was cooled at 0° C. and treated with 1-(3-dimethylaminopropyl)-3-ethylcarbodiimide hydrochloride (1.08 g, 5.65 mmol). The cloudy reaction mixture was warmed to room temperature and stirred for 16 h, then diluted with ethyl acetate (125 mL) and washed with 5% citric acid solution (25 mL). The organic phase was washed with satd NaHCO₃ solution (25 mL), 5% aqueous NaCl solution (2×25 mL), and brine (25 mL), dried over Na₂SO₄, filtered and concentrated. The crude product was purified by silica gel chromatography eluting with 0-5% MeOH in CH₂Cl₂ to provide 806 mg (81%) of 3-bromo-2-methyl-benzamide.

A solution of 3-bromo-2-methyl-benzamide (450 mg, 2.10 mmol) in THF (25 mL) was treated with borane in THF (8.40 μL, 1 M in THF, 8.40 mmol). The reaction mixture was heated at reflux for 14 h, then cooled to 0° C. and quenched by the careful dropwise addition of MeOH. After stirring for an additional 10 min at room temperature, the r×n mixture was concentrated, re-dissolved in 10% MeOH in CH₂Cl₂, and treated with 1 M HCl solution (6 mL). This mixture was stirred vigorously for 10 min, then cooled at 0° C. and made basic by the addition of 2.5 M NaOH solution. The resulting mixture was diluted with water and dichloromethane, and the organic phase was washed with water and brine, dried over Na₂SO₄, filtered and concentrated to give 426 mg of 3-bromo-2-methyl-benzylamine as a cloudy colorless oil that was used without further purification.

3-Bromo-2-fluoro-benzylamine was prepared by a procedure analogous to that described in the above example using 3-bromo-2-fluoro-benzoic acid as starting material.

3-Bromo-2-trifluoromethoxy-benzylamine was prepared by a procedure analogous to that described in the above example using 3-bromo-2-trifluoromethoxy-benzoic acid as starting material. 3-Bromo-2-trifluoromethoxy-benzoic acid was synthesized according to literature precedent (Schlosser, M.; Castagnetti, E. Eur. J. Org. Chem. 2001, 3991-3997).

3-Bromo-2-chloro-benzylamine was prepared by a procedure analogous to that described in the above example using 3-bromo-2-chloro-benzoic acid as starting material. 3-Bromo-2-chloro-benzoic acid was synthesized according to literature precedent (Gohier, F.; Mortier, J. J. Org. Chem. 2003, 68, 2030-2033).

### Synthesis of (5-Bromo-pyridin-3-yl)-methylamine

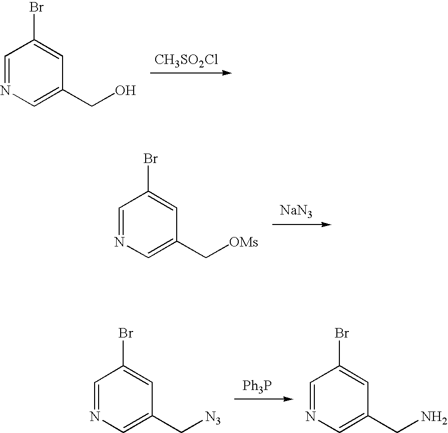

To a solution of (5-bromo-pyridin-3-yl)-methanol (2.75 g, 14.6 mmol) and Et₃N (3.10 mL, 22.2 mmol) in DCM (75 mL) at −20° C. under N₂ was added methanesulfonyl chloride (1.70 mL, 22.2 mmol) dropwise. After 45 min the reaction was allowed to warm to room temperature and diluted with DCM (75 mL). The reaction mixture was washed with water (75 mL), sat. NaHCO₃ (2×75 mL) and brine before drying over Na₂SO₄. Concentration in vacuo afforded crude methanesulfonic acid 5-bromo-pyridin-3-ylmethyl ester (4.21 g) as an oil. The crude material was used in the next step without purification.

To a solution of crude methanesulfonic acid 5-bromo-pyridin-3-ylmethyl ester (4.20 g) in DMF (60 mL) was added NaN₃ (10.0 g, 153.8 mmol). The mixture was stirred under N₂ overnight then diluted with water. The mixture was extracted with EtOAc (2×300 mL) and the combined organic layers washed with water before drying over Na₂SO₄. The solution was concentrated in vacuo to afford crude 3-azidomethyl-5-bromo-pyridine (2.04 g) as a dark brown oil. This crude material was used directly in the next step.

To a solution of crude 3-azidomethyl-5-bromo-pyridine (2.04 g) in THF (50 μL) and water (1 mL) was added triphenylphosphine (5.02 g, 19.1 mmol). The mixture was heated at reflux under N₂ for 2 h before cooling to room temperature and concentrating in vacuo. The residue was purified by column chromatography using an ISCO combi-flash cartridge (silica gel, 100:0 to 30:70 DCM/10% NH₄OH in MeOH) to afford (5-bromo-pyridin-3-yl)-methylamine (0.97 g)

### Example 16

### Synthesis of N-{[3′-(aminomethyl)-2-methylbiphenyl-3-yl]methyl}-5-nitro-4-(4,5,7,8-tetrahydroimidazo[4,5-d]azepin-6(1H)-yl)pyrimidin-2-amine

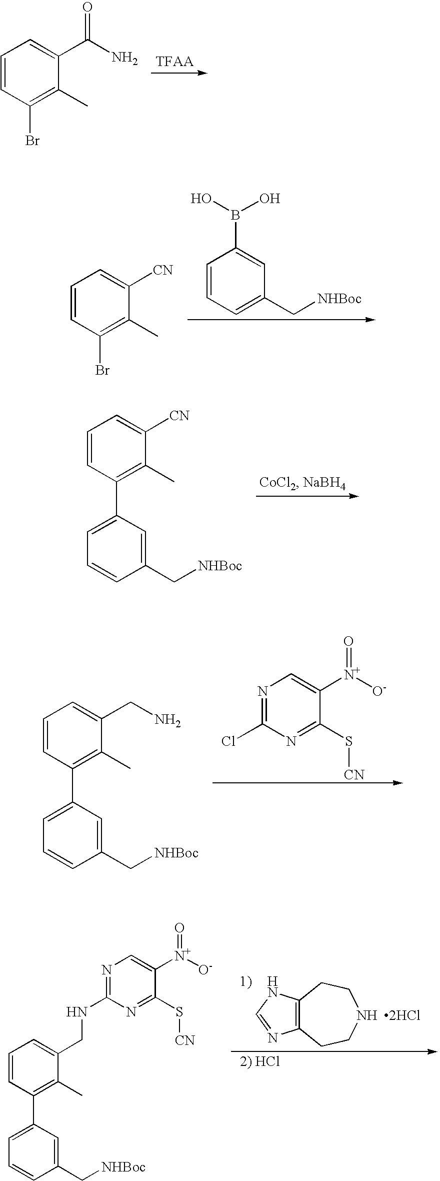

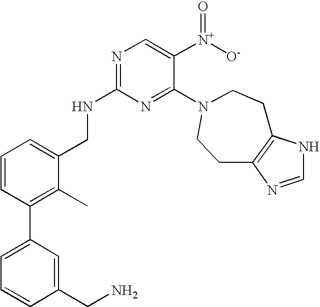

A solution of 3-bromo-2-methyl-benzamide (500 mg, 2.340 mmol) and pyridine (0.60 mL, 7.42 mmol) in dichloromethane (10 mL) was cooled to 0° C. and treated with trifluoroacetic anhydride (1.0 mL, 7.08 mmol). The reaction mixture was warmed to room temperature and stirred for 15 h, then partitioned between ethyl acetate (100 mL) and 1 M HCl solution (25 mL). The organic phase was washed with satd NaHCO₃ solution (25 mL) and brine (25 mL), dried over Na₂SO₄, filtered and concentrated to afford 423 mg (92%) of 3-bromo-2-methyl-benzonitrile as a pale yellow oil that was carried on without further purification.

To a mixture of 3-bromo-2-methyl-benzonitrile (400 mg, 2.04 mmol), [3-N-boc-aminomethyl)phenyl]boronic acid (768 mg, 3.06 mmol), tetrakis(triphenylphosphine)palladium (236 mg, 0.204 mmol), and sodium carbonate (649 mg, 6.12 mmol) was added dimethoxyethane (16 mL) and water (1.6 mL). The reaction mixture was sealed under N₂ and heated at 120° C. for 2 h. The reaction mixture was partitioned between ethyl acetate (125 mL) and 5% NaCl solution (40 mL). The organic phase was washed with brine, dried over Na₂SO₄, filtered and concentrated. The residue was purified by silica gel chromatography eluting with 5-15% ethyl acetate in hexanes to furnish 604 mg (92%) of (3′-cyano-2′-methyl-biphenyl-3-ylmethyl)-carbamic acid tert-butyl ester, m/z 323.5 (M+H)⁺.

A solution of (3′-cyano-2′-methyl-biphenyl-3-ylmethyl)-carbamic acid tert-butyl ester (100 mg, 0.31 mmol) and cobaltous chloride hexahydrate (111 mg, 0.47 mmol) in 2:1 THF/H₂O (3 mL) was cooled to 0° C. and treated with sodium borohydride (59 mg, 1.55 mmol), portionwise over several minutes. The reaction mixture was warmed to room temperature and stirred for 1 h. After the addition of 1 mL of conc NH₄OH, the mixture was stirred for 5 min and then filtered, washing the collected solids with 2:1 THF/H₂O (20 mL). The filtrates were concentrated, and the residue was partitioned between dichloromethane (15 mL) and water (5 mL). The aqueous phase was extracted with dichloromethane (2×15 mL), and the combined organic phases were washed with brine, dried over Na₂SO₄, filtered and concentrated to furnish 74 mg (73%) of (3′-aminomethyl-2′-methyl-biphenyl-3-ylmethyl)-carbamic acid tert-butyl ester as an off-white solid, m/z 327.6 (M+H)⁺. (Osby, J. O.; Heinzman, S. W.; Ganem, B. J. Am. Chem. Soc. 1986, 108, 67-72).

To a solution of (3′-aminomethyl-2′-methyl-biphenyl-3-ylmethyl)-carbamic acid tert-butyl ester (76 mg, 0.23 mmol) and diisopropylethylamine (0.045 mL, 0.26 mmol) in dichloromethane (2.0 mL) was added 2-chloro-5-nitro-4-thiocyanato-pyrimidine (50 mg, 0.23 mmol). The r×n mixture was stirred at room temperature for 72 h, then diluted with 5% MeOH in CH₂Cl₂ (30 mL) and washed with 1 M HCl solution (8 μL). The organic phase was washed with satd NaHCO₃ solution and brine, dried over Na₂SO₄, filtered and concentrated. The crude product was purified by silica gel chromatography eluting with 15-30% ethyl acetate in hexanes to afford 74 mg (63%) of {2′-methyl-3′-[(5-nitro-4-thiocyanato-pyrimidin-2-ylamino)-methyl]-biphenyl-3-ylmethyl}-carbamic acid tert-butyl ester, m/z 507.5 (M+H)⁺.

To a solution of 1,4,5,6,7,8-hexahydro-imidazo[4,5-d]azepine, di-HCl salt (37 mg, 0.18 mmol) and diisopropylethylamine (0.102 mL, 0.58 mmol) in 3:1 dichloromethane/N,N-dimethylformamide (2.0 mL) was added {2′-methyl-3′-[(5-nitro-4-thiocyanato-pyrimidin-2-ylamino)-methyl]-biphenyl-3-ylmethyl}-carbamic acid tert-butyl ester (74 mg, 0.15 mmol). The r×n mixture was stirred at room temperature for 7.5 h, then diluted with ethyl acetate (35 mL) and washed with 5% NaCl solution (3×12 mL) and brine (15 mL), dried over Na₂SO₄, filtered and concentrated. The crude product was purified by silica gel chromatography eluting with 0-5% MeOH in CH₂Cl₂ to give 63 mg (74%) of (2′-methyl-3′-{[5-nitro-4-(4,5,7,8-tetrahydro-1H-imidazo[4,5-d]azepin-6-yl)-pyrimidin-2-ylamino]-methyl}-biphenyl-3-ylmethyl)-carbamic acid tert-butyl ester, m/z 585.8 (M+H)⁺.

A solution of (2′-methyl-3′-{[5-nitro-4-(4,5,7,8-tetrahydro-1H-imidazo[4,5-d]azepin-6-yl)-pyrimidin-2-ylamino]-methyl}-biphenyl-3-ylmethyl)-carbamic acid tert-butyl ester (63 mg, 0.11 mmol) in dichloromethane (4.0 mL) was treated with 4 M HCl in dioxane (0.135 mL, 0.54 mmol). The reaction mixture was stirred at room temperature for 4 h and then filtered, washing the solid with dichloromethane. The solid was dried in a vacuum oven (50° C.) to yield 51 mg (85%) of N-{[3′-(aminomethyl)-2-methylbiphenyl-3-yl]methyl}-5-nitro-4-(4,5,7,8-tetrahydroimidazo[4,5-d]azepin-6(1H)-yl)pyrimidin-2-amine as a yellow solid, isolated as the di-HCl salt, m/z 485.9 (M+H)⁺.

The compounds according to Examples 55 and 57-63 as presented in Table 1 may be prepared by a procedure analogous to that described above in Example 16. In some cases, trifluoroacetic acid was substituted for HCl in dioxane in the final boc deprotection step.

The compounds according to Examples 64-67 as presented in Table 1 may prepared by a procedure analogous to that described above in Example 16 by using 3-bromo-2-chloro-benzonitrile as starting material

The compound according to Example 56 was prepared by a procedure analogous to that described above in Example 16 except (4-aminomethyl-cyclohexylmethyl)-urea was used.

### Synthesis of (4-aminomethyl-cyclohexylmethyl)-urea

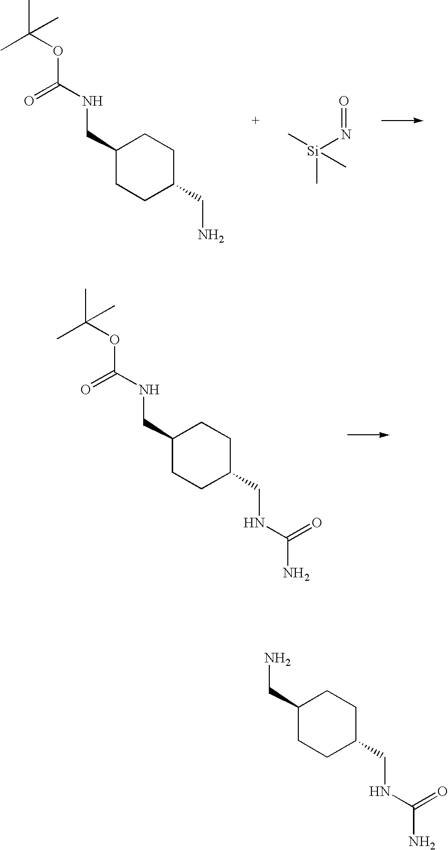

To a solution of (4-aminomethyl-cyclohexylmethyl)-carbamic acid tert-butyl ester (200 mg, 0.83 mmol) in dichloromethane (2.0 mL) was added the trimethylsilylisocyanate (890 μL, 6.6 mmol) and the solution was heated at 80° C. for 16 hours. The solution was concentrated, diluted with dichloromethane, filtered and washed with dichloromethane to afford 165 mg (70%) of (4-urcidomethyl-cyclohexylmethyl)-carbamic acid tert-butyl ester.

To a solution of the (4-ureidomethyl-cyclohexylmethyl)-carbamic acid tert-butyl ester (34 mg, 0.12 mmol) in dichloromethane (0.75 mL) was added trifluoroacctic acid (0.35 mL) and the solution was stirred for 16 hours. The solution was concentrated to afford 22 mg (100%) of (4-aminomethyl-cyclohexylmethyl)-urea.

### Synthesis of (4-hydroxy-cyclohexylmethyl)-carbamic acid tert-butyl ester

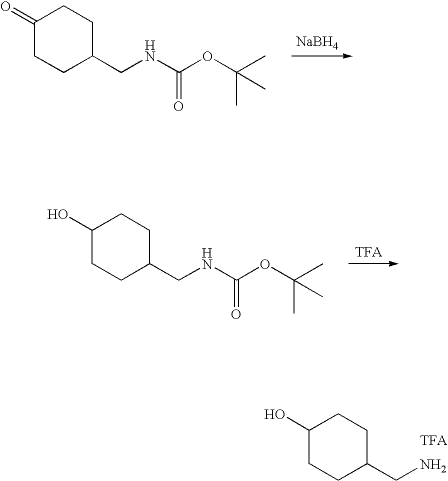

A solution of (4-oxo-cyclohexylmethyl)-carbamic acid tert-butyl ester (2.00 g, 8.80 mmol) in methanol (150 mL) was cooled at 0° C. and treated with sodium borohydride (1.33 g, 35.2 mmol). The reaction mixture was warmed to room temperature and stirred for 18 h, and then quenched by the addition of ice (20 g) and satd NH₄Cl solution (50 mL). This mixture was extracted with ethyl acetate (3×100 mL), and the combined organic phases were dried over MgSO₄, filtered and concentrated to yield 2.0 g (99%) of (4-hydroxy-cyclohexylmethyl)-carbamic acid tert-butyl ester as a white foam, isolated as a 4:1 mixture of cis:trans isomers by proton NMR analysis.

A solution of (4-hydroxy-cyclohexylmethyl)-carbamic acid tert-butyl ester (432 mg, 1.88 mmol) in dichloromethane (6.0 mL) was treated with trifluoroacetic acid (1.40 mL, 18.2 mmol). The reaction mixture was stirred at room temperature for 7 h and then concentrated. The residue was redissolved in 10% MeOH in CH₂Cl₂ and concentrated to provide 4-aminomethyl-cyclohexanol TFA salt as an oily solid that was used without further purification.

### Example 17

### Synthesis of N-[4-({2-[(3′-Aminomethyl-2-methyl-biphenyl-3-ylmethyl)-amino]-5-nitro-pyrimidin-4-ylamino}-methyl)-cyclohexyl]-acetamide

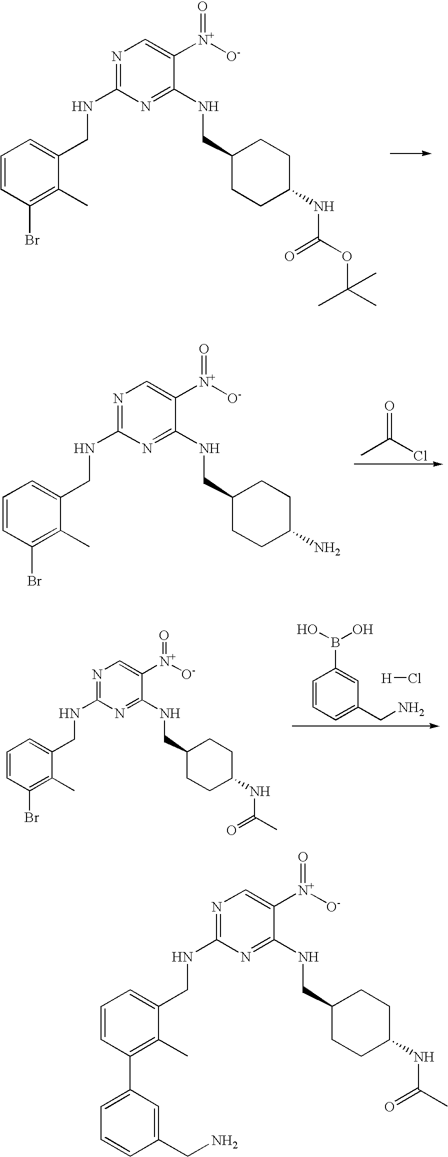

To a solution of (4-{[2-(3-bromo-2-methyl-benzylamino)-5-nitro-pyrimidin-4-ylamino]-methyl}-cyclohexyl)-carbamic acid tert-butyl ester (40 mg, 0.073 mmol) in dichloromethane (0.4 mL) was added the trifluoroacetic acid (0.2 mL) and the solution was stirred for 4 b. The solution was concentrated to afford 32.7 mg (66%) of N⁴-(4-amino-cyclohexylmethyl)-N²-(3-bromo-2-methyl-benzyl)-5-nitro-pyrimidine-2,4-diamine as the bis-trifluoroacetate salt.

To a solution of the N⁴-(4-amino-cyclohexylmethyl)-N²-(3-bromo-2-methyl-benzyl)-5-nitro-pyrimidine-2,4-diamine bis-trifluoroacetate salt (93 mg, 0.138 mmol) in dichloromethane (0.75 mL) was added the PS-DIEA (230 mg, 0.828 mmol) and the mixture was shaken for 1 hour. The solution was filtered and concentrated to afford the free base. To a solution of the free base (62 mg, 0.138 mmol) in dichloromethane (0.75 mL) was added acetyl chloride (19.6 μL, 0.276 mmol) and diisopropylethylamine (72 μL, 0.414 mmol) and the solution was stirred for 1 h. The reaction mixture was diluted with dichloromethane (2 mL), and saturated aqueous NaHCO₃ (2 mL) was added. The phases were separated and the aqueous phase was extracted with dichloromethane (3 mL). The organic phases were combined, dried over Na₂SO₄, and concentrated to afford 68 mg (82%) of N-(4-{[2-(3-bromo-2-methyl-benzylamino)-5-nitro-pyrimidin-4-ylamino]-methyl}-cyclohexyl)-acetamide.

To a mixture of N-(4-{[2-(3-bromo-2-methyl-benzylamino)-5-nitro-pyrimidin-4-ylamino]-methyl}-cyclohexyl)-acetamide (55 mg, 0.113 mmol), (3-aminomethylphenyl)boronic acid HCl (32 mg, 0.169 mmol), tetrakis(triphenylphosphine)palladium (13 mg, 0.011 mmol), and potassium carbonate (62 mg, 0.451 mmol) was added dimethoxyethane (0.8 mL) and water (0.08 mL). The reaction mixture was sealed under N₂ and heated at 190° C. in the microwave for 5 minutes. The reaction mixture was purified directly by 1 μm preparative plate silica gel chromatography eluting with 10% MeOH in CH₂Cl₂ to furnish 4.2 mg (7.2%) of N-[4-({2-[(3′-aminomethyl-2-methyl-biphenyl-3-ylmethyl)-amino]-5-nitro-pyrimidin-4-ylamino}-methyl)-cyclohexyl]-acetamide, m/z 518.7 (M+H)⁺.

The compound according to Example 68 as presented in Table 1 may be prepared by a procedure analogous to that described above in Example 17 using (4-{[2-(3-bromo-benzylamino)-5-nitro-pyrimidin-4-ylamino]-methyl}-cyclohexyl)-carbamic acid tert-butyl ester as starting material.

The compound according to Example 69 as presented in Table 1 may prepared by a procedure analogous to that described above in Example 17 except methanesulfonyl chloride was used.

The compound according to Example 70 as presented in Table 1 may be prepared by a procedure analogous to that described above in Example 17 except (trans-4-{[2-(3-bromo-2-methyl-benzylamino)-5-nitro-pyrimidin-4-ylamino]-methyl}-cyclohexylmethyl)-carbamic acid tert-butyl ester was used as starting material.

### Example 13

### Synthesis of N⁴-{[trans-4-(4-acetylpiperazin-1-yl)cyclohexyl]methyl}-N²-{[3-(aminomethyl)-2-methylbiphenyl-3-yl]methyl}-5-nitropyrimidine-2,4-diamine

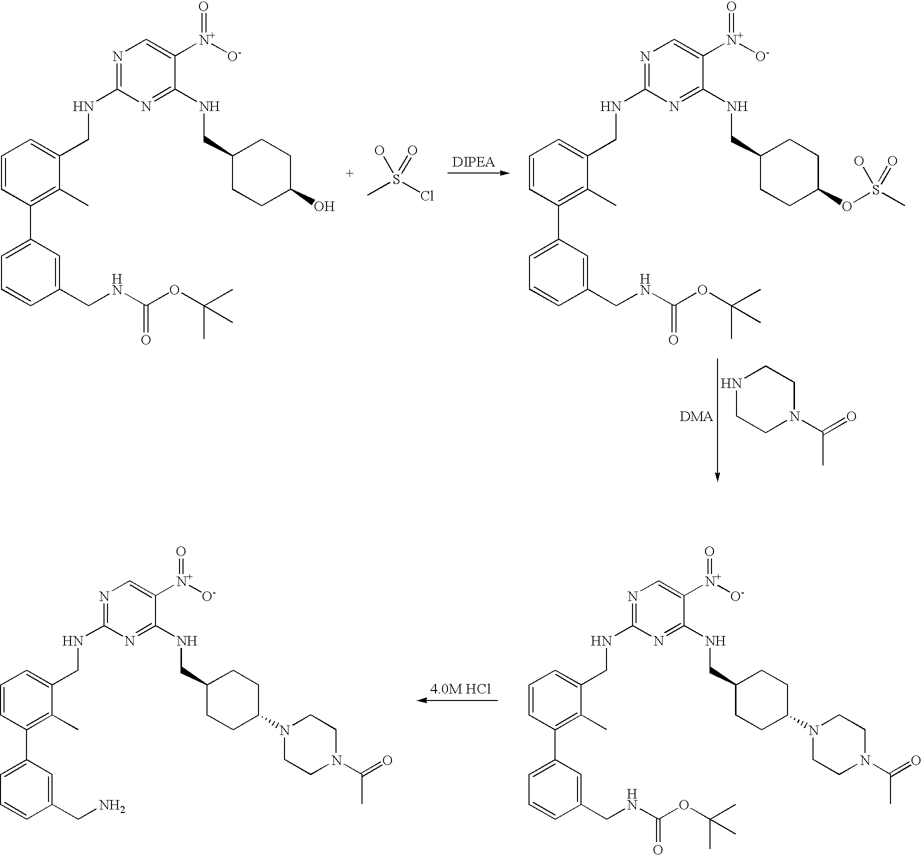

To a solution of [3′-({4-[(4-hydroxy-cyclohexylmethyl)-amino]-5-nitro-pyrimidin-2-ylamino}-methyl)-2′-methyl-biphenyl-3-ylmethyl]-carbamic acid tert-butyl ester (277 mg, 0.480 mmol) in CH₂Cl₂ (15 mL) at 5° C. was added diisopropylethylamine (0.250 mL, 1.44 mmol) and methanesulfonyl chloride (56 μL, 0.720 mmol). The reaction mixture was stirred at room temperature for 2 h and then it was quenched with cold water (10 mL) and warmed to room temperature. The mixture was partitioned between CH₂Cl₂ and water. The organic layers was combined, washed with brine, dried over Na₂SO₄. After filtration, the filtrate was concentrated in vacuo to afford a yellow residue. The residue was then purified by silica gel chromatography eluting with 1% MeOH in CH₂Cl₂ to afford 300 mg (95%) of methanesulfonic acid 4-[(2-{[3′-(tert-butoxycarbonylamino-methyl)-2-methyl-biphenyl-3-ylmethyl]-amino}-5-nitro-pyrimidin-4-ylamino)-methyl]-cyclohexyl ester as a pale-yellow solid.

Methanesulfonic acid, 4-[(2-{[3′-(tert-butoxycarbonylamino-methyl)-2-methyl-biphenyl-3-ylmethyl]-amino}-5-nitro-pyrimidin-4-ylamino)-methyl]-cyclohexyl ester (80 mg, 0.120 mmol), and 1-piperazin-1-yl-ethanone (78 mg, 0.610 mmol) were mixed in dimethylacetamide (0.300 mL) and heated at 100° C. for 8 h. The mixture was then dissolved in MeOH and purified by preparative HPLC to afford 10 mg (12%) {3′-[(4-{[4-(4-acetyl-piperazin-1-yl)-cyclohexylmethyl]-amino}-5-nitro-pyrimidin-2-ylamino)-methyl]-2′-methyl-biphenyl-3-ylmethyl}-carbamic acid tert-butyl ester as a light brown solid.

A solution of {3′-[(4-{[4-(4-acetyl-piperazin-1-yl)-cyclohexylmethyl]-amino}-5-nitro-pyrimidin-2-ylamino)-methyl]-2′-methyl-biphenyl-3-ylmethyl}-carbamic acid tert-butyl ester (10 mg, 0.020 mmol) in dichloromethane (1 mL) was treated with 4.0 M HCl (37 μL, 0.150 mmol). The reaction mixture was stirred at room temperature for 1 h and a white precipitate formed. The precipitate was isolated and dried in vacuo to afford 8 mg (94%) of N⁴-{[trans-4-(4-acetylpiperazin-1-yl)cyclohexyl]methyl)-N²— ([3′-(aminomethyl)-2-methylbiphenyl-3-yl]methyl}-5-nitropyrimidine-2,4-diamine (HCl salt) as pale yellow solid, m/z 587.1 (M+H)⁺.

The compounds according to Examples 71-73 as presented in Table 1 may be prepared by a procedure analogous to that described in above Example 18.

### Example 19

### Synthesis of N²-{[3′-({[4-({[trans-4-(aminomethyl)cyclohexyl]methyl}-amino)-5-nitropyrimidin-2-yl]amino}methyl)-2′-methylbiphenyl-3-yl]methyl}-glycinamide

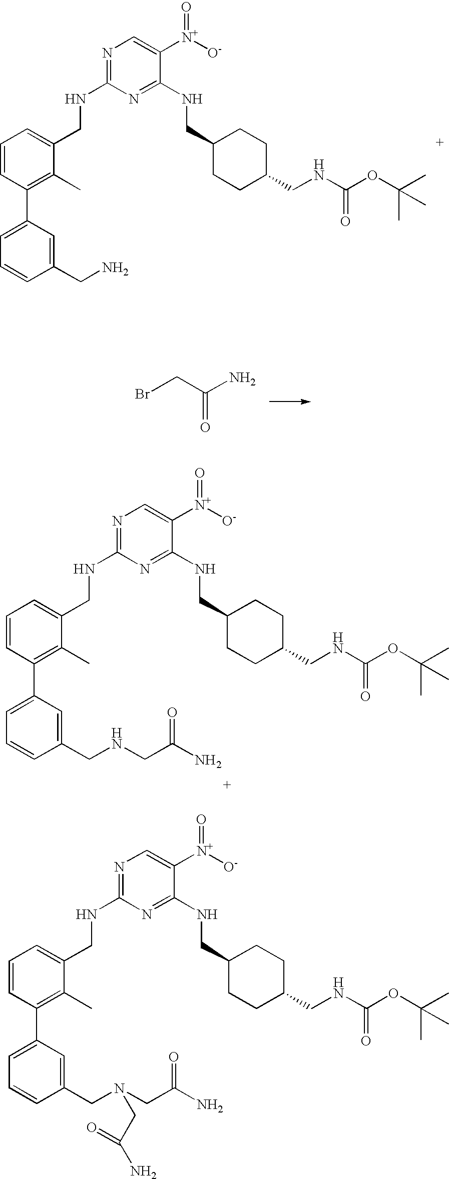

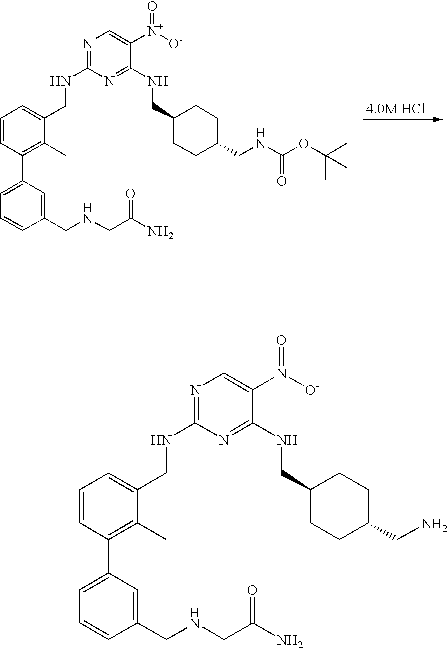

To a solution of N²-{[3′-({[4-({[trans-4-(aminomethyl)cyclohexyl]methyl}-amino)-5-nitropyrimidin-2-yl]amino}methyl)-2′-methylbiphenyl-3-yl]methyl}-glycinamide (0.100 g, 0.170 mmol) in CH₂Cl₂ (5 mL) was added 2-bromo-acetamide (26 mg, 0.190 mmol) followed by diisopropylethylamine (31 μL, 0.170 mmol). The reaction mixture was stirred at room temperature for 8 h. The reaction mixture was diluted with EtOAc (20 mL) and washed with water (5 mL). The organic layers were separated, combined, dried over Na₂SO₄. After filtration, the filtrate was concentrated to afford a residue in vacuo. The residue was purified using preparative silica gel chromatography (5% MeOH/CH₂Cl₂) to afford 70 mg (64%) of (4-{[2-({3′-[(carbamoylmethyl-amino)-methyl]-2-methyl-biphenyl-3-ylmethyl}-amino)-5-nitro-pyrimidin-4-ylamino]-methyl}-cyclohexylmethyl)-carbamic acid tert-butyl ester as pale-yellow solid and 35 mg (29%) of (4-{[2-({3′-[(bis-carbamoylmethyl-amino)-methyl]-2-methyl-biphenyl-3-ylmethyl}-amino)-5-nitro-pyrimidin-4-ylamino]-methyl}-cyclohexylmethyl)-carbamic acid tert-butyl ester as a pale-yellow solid.

A solution of (4-{[2-({3′-[(carbamoylmethyl-amino)-methyl]-2-methyl-biphenyl-3-ylmethyl}-amino)-5-nitro-pyrimidin-4-ylamino]-methyl}-cyclohexylmethyl)-carbamic acid tert-butyl ester (70 mg, 0.110 mmol) in dichloromethane (1 mL) was treated with 4.0 M HCl (0.270 mL, 1.08 mmol). The reaction mixture was stirred at room temperature for 1 h and a pale-yellow precipitate formed. The crude product was isolated after an aqueous workup in which the excess acid was neutralized with satd NaHCO₃ solution, and the desired product was extracted with dichloromethane. The solvent was removed in vacuo to afford 15 mg (25%) of N²-{[3′-({[4-({[trans-4-(aminomethyl)cyclohexyl]methyl}-amino)-5-nitropyrimidin-2-yl]amino}methyl)-2′-methylbiphenyl-3-yl]methyl}-glycinamide as a pale yellow solid, m/z 547.1 (M+H)⁺ BI00612671BS.

The compound according to Example 74 as presented in Table 1 may be prepared by application of an amine deprotection procedure analogous to that described above in Example 19 to (4-{[2-({3′-[(bis-carbamoylmethyl-amino)-methyl]-2-methyl-biphenyl-3-ylmethyl}-amino)-5-nitro-pyrimidin-4-ylamino]-methyl}-cyclohexylmethyl)-carbamic acid tert-butyl ester.

### Example 20

### Synthesis of 2-([[3′-(aminomethyl)-2-methylbiphenyl-3-yl]methyl]amino)-4-(4,5,7,8-tetrahydroimidazo[4,5-d]azepin-6(1H)-yl)pyrimidine-5-carbonitrile

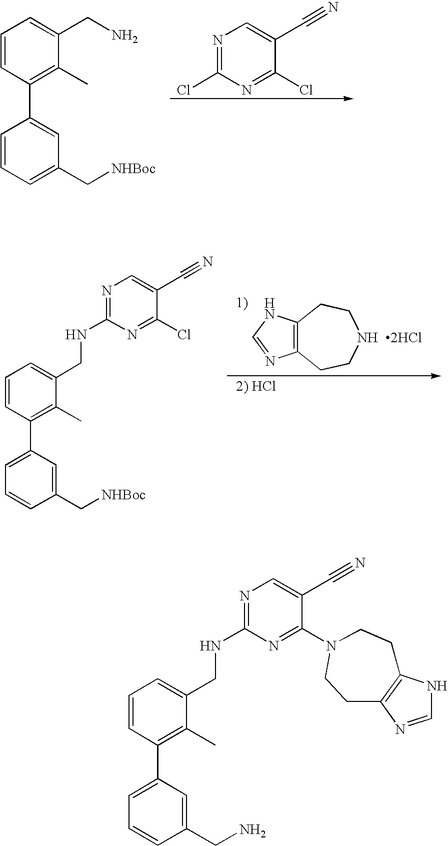

To a solution of (3′-aminomethyl-2′-methyl-biphenyl-3-ylmethyl)-carbamic acid tert-butyl ester (281 mg, 0.86 mmol) and diisopropylethylamine (0.165 mL, 0.95 mmol) in dichloromethane (5.0 mL) was added 2,4-dichloro-pyrimidine-5-carbonitrile (150 mg, 0.86 mmol). The r×n mixture was stirred at room temperature for 70 h, then adsorbed directly onto silica gel and purified by silica gel chromatography eluting with 20-45% ethyl acetate in hexanes to afford 254 mg of a colorless oil, identified as a 1:1 mixture of the desired product and the regioisomeric 4-addition product. This mixture was further purified by reverse phase semi-prep HPLC using 60% acetonitrile/water (0.1% TFA) as eluent to give 149 mg of {3′-[(4-chloro-5-cyano-pyrimidin-2-ylamino)-methyl]-2′-methyl-biphenyl-3-ylmethyl}-carbamic acid tert-butyl ester as an off-white solid, m/z 486.0 (M+Na)⁺.

To a solution of 1,4,5,6,7,8-hexahydro-imidazo[4,5-d]azepine, di-HCl salt (41 mg, 0.19 mmol) and diisopropylethylamine (0.141 mL, 0.81 mmol) in 3:1 dichloromethane/N,N-dimethylformamide (2.0 mL) was added {3′-[(4-chloro-5-cyano-pyrimidin-2-ylamino)-methyl]-2′-methyl-biphenyl-3-ylmethyl}-carbamic acid tert-butyl ester (75 mg, 0.16 mmol). The r×n mixture was stirred at room temperature for 84 h, then diluted with ethyl acetate (50 mL) and washed with 5% NaCl solution (2×15 mL) and brine (15 mL), dried over Na₂SO₄, filtered and concentrated. The crude product was purified by silica gel chromatography eluting with 1-8% MeOH in CH₂Cl₂ to give 67 mg (73%) of (3′-{[5-cyano-4-(4,5,7,8-tetrahydro-1H-imidazo[4,5-d]azepin-6-yl)-pyrimidin-2-ylamino]-methyl}-2′-methyl-biphenyl-3-ylmethyl)-carbamic acid tert-butyl ester, m/z 565.1 (M+H)⁺.

A solution of (3′-{[5-cyano-4-(4,5,7,8-tetrahydro-1H-imidazo[4,5-d]azepin-6-yl)-pyrimidin-2-ylamino]-methyl}-2′-methyl-biphenyl-3-ylmethyl)-carbamic acid tert-butyl ester (67 mg, 0.12 mmol) in dichloromethane (3.0 mL) was treated with 4 M HCl in dioxane (0.149 mL, 0.60 mmol). The reaction mixture was stirred at room temperature for 6 h and then filtered, washing the solid with 5% MeOH in dichloromethane and dichloromethane. The gummy solid was concentrated and then purified by reverse phase prep HPLC using acetonitrile/water (0.1% formic acid) gradient elution to afford 47 mg (85%) of 2-({[3′-(aminomethyl)-2-methylbiphenyl-3-yl]methyl}amino)-4-(4,5,7,8-tetrahydroimidazo[4,5-d]azepin-6(1H)-yl)pyrimidine-5-carbonitrile as pale yellow solid, isolated as the di-formate salt, m/z 465.1 (M+H)⁺.

The compound according to Example 75 as presented in Table 1 may be prepared using a procedure analogous to that described above in Example 20.

TABLE 1

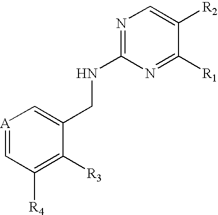

| Ex # | R₁ | R₂ | R₃ | R₄ | A | MS |
|---|---|---|---|---|---|---|
| 1 | 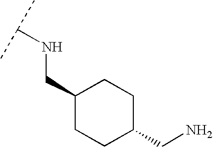 | NO₂ | H | 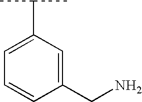 | C | 477(M + H)⁺ |
| 2 | 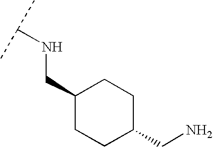 | NO₂ | H | 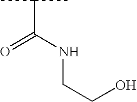 | C | 459(M + H)⁺ |
| 3 | 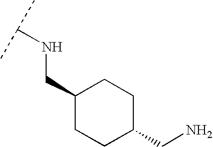 | NO₂ | H | 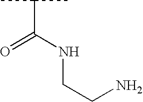 | C | 458(M + H)⁺ |
| 4 | 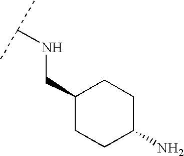 | NO₂ | CH₃ | 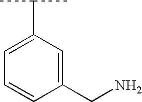 | C | 475(M − H)⁻ |
| 5 | 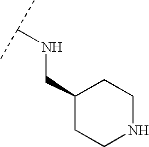 | NO₂ | CH₃ | 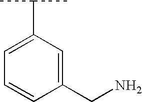 | C | 463(M + H)⁺ |
| 6 | 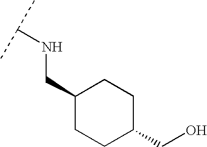 | NO₂ | H | 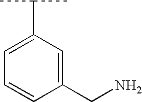 | C | 478(M + H)⁺ |
| 7 | 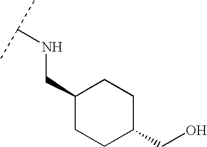 | NO₂ | H | 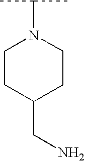 | C | 484(M + H)⁺ |
| 8 | 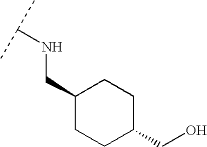 | NO₂ | H | 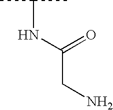 | C | 444(M + H)⁺ |
| 9 | 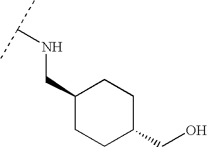 | NO₂ | CH₃ | 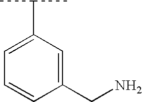 | C | 492(M + H)⁺ |
| 10 | 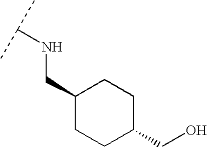 | NO₂ | CH₃ | 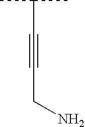 | C | 440(M + H)⁺ |
| 11 | 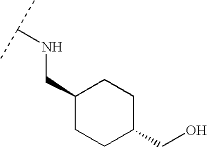 | NO₂ | CH₃ | 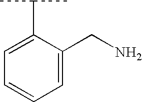 | C | 492(M + H)⁺ |
| 12 | 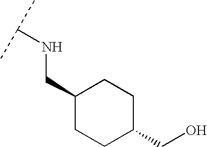 | NO₂ | CH₃ | 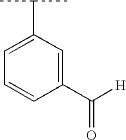 | C | 490(M + H)¹ |
| 13 | 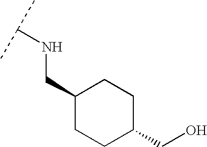 | NO₂ | CH₃ | 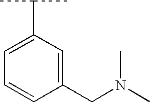 | C | 520(M + H)⁺ |
| 14 | 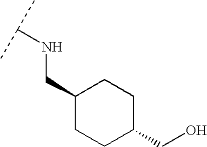 | NO₂ | CH₃ | 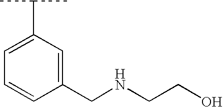 | C | 535(M + H)⁺ |
| 15 | 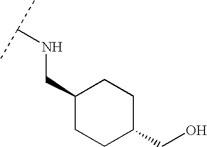 | NO₂ | CH₃ | 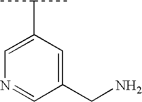 | C | 492(M + H)⁺ |
| 16 | 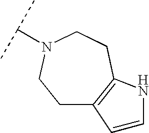 | NO₂ | CH₃ | 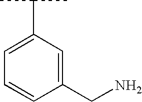 | C | 486(M + H)⁺ |
| 17 | 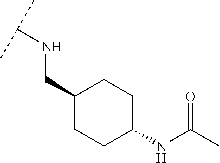 | NO₂ | CH₃ | 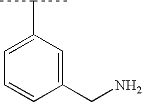 | C | 519(M + H)⁺ |
| 18 | 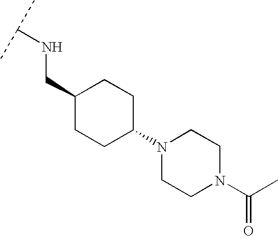 | NO₂ | CH₃ | 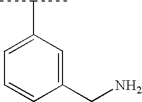 | C | 587(M + H)⁺ |
| 19 | 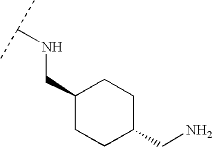 | NO₂ | CH₃ | 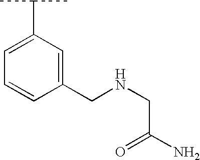 | C | 547(M + H)⁺ |
| 20 | 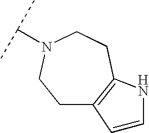 | CN | CH₃ | 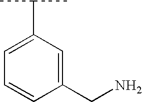 | C | 465(M + H)⁺ |
| 21 | 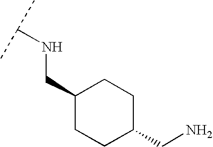 | NO₂ | H | 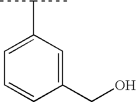 | C | 477(M + H)⁺ |
| 22 | 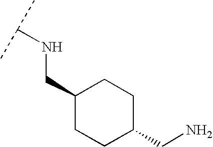 | NO₂ | H | 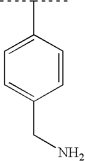 | C | 476(M + H)⁺ |
| 23 | 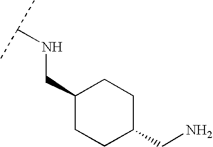 | NO₂ | H | 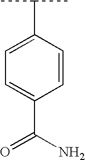 | C | 490(M + H)⁺ |
| 24 | 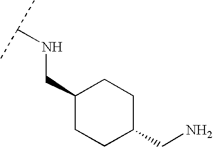 | NO₂ | H | 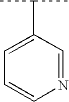 | C | 448(M + H)⁺ |
| 25 | 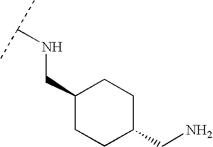 | NO₂ | H | 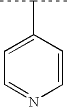 | C | 448(M + H)⁺ |
| 26 | 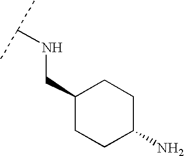 | NO₂ | H | 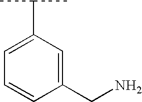 | C | 463(M + H)⁺ |
| 27 | 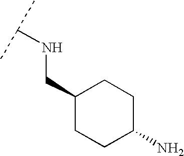 | NO₂ | H | 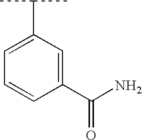 | N | 477(M + H)⁺ |
| 28 | 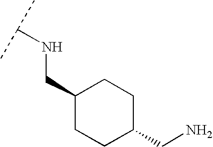 | NO₂ | H | 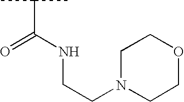 | C | 528(M + H)⁺ |
| 29 | 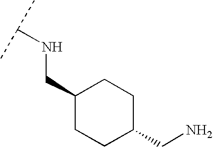 | NO₂ | H | 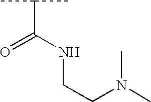 | C | 486(M + H)⁺ |
| 30 | 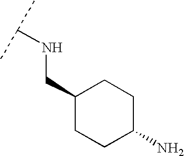 | NO₂ | Cl | 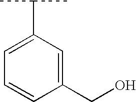 | C | 499(M + H)⁺ |
| 31 | 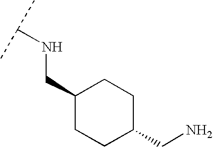 | NO₂ | CH₃ | 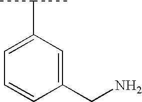 | C | 489(M − H)⁻ |
| 32 | 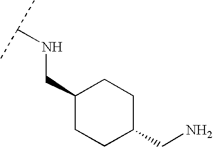 | NO₂ | CH₃ | 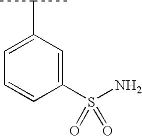 | C | 540(M + H)⁺ |
| 33 | 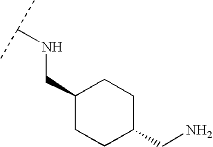 | NO₂ | CH₃ | 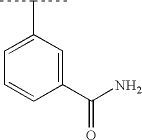 | C | 505(M + H)⁺ |
| 34 | 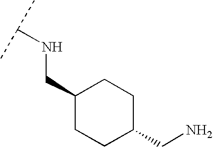 | NO₂ | CH₃ | 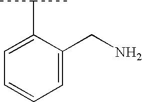 | C | 491(M + H)⁺ |
| 35 | 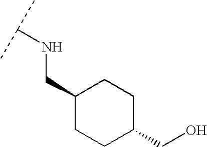 | NO₂ | H | 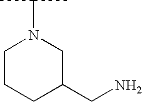 | C | 484(M + H)⁺ |
| 36 | 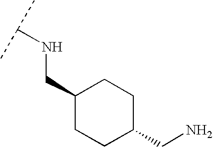 | NO₂ | H | 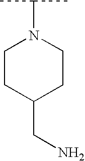 | C | 483(M + H)⁺ |
| 37 | 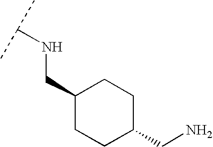 | NO₂ | H | 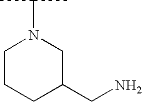 | C | 483(M + H)⁺ |
| 38 | 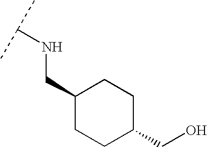 | NO₂ | H | 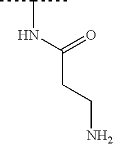 | C | 458(M + H)⁺ |
| 39 | 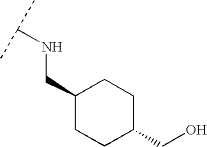 | NO₂ | CH₃ | 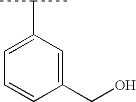 | C | 493(M + H)⁺ |
| 40 | 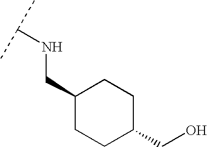 | NO₂ | CH₃ | 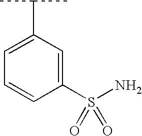 | C | 542(M + H)⁺ |
| 41 | 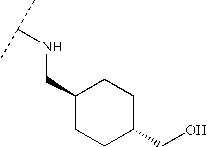 | NO₂ | CH₃ | 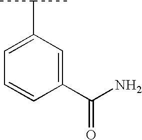 | C | 506(M + H)⁺ |
| 42 | 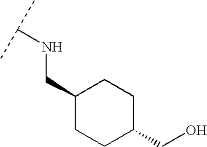 | NO₂ | CH₃ | 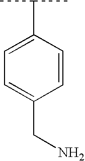 | C | 492(M + H)⁺ |
| 43 | 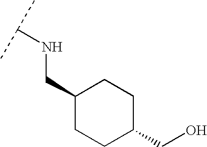 | NO₂ | CH₃ | 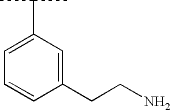 | C | 548(M +CH₃CN)¹ |
| 44 | 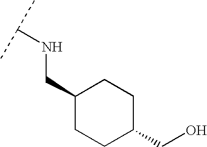 | NO₂ | F | 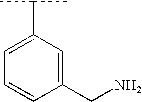 | C | 496(M + H)⁺ |
| 45 | 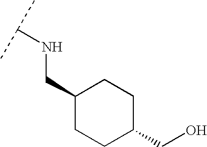 | NO₂ | OCF₃ | 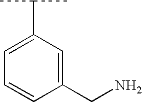 | C | 562(M + H)⁺ |
| 46 | 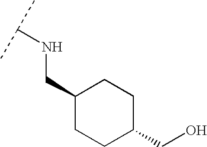 | NO₂ | Cl | 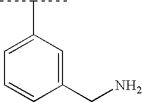 | C | 512(M + H)⁺ |
| 47 | 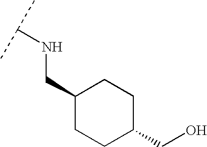 | NO₂ | CH₃ | 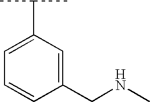 | C | 506(M + H)⁺ |
| 48 | 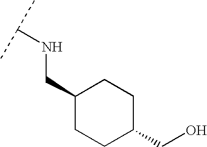 | NO₂ | CH₃ | 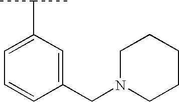 | C | 560(M + H)⁺ |
| 49 | 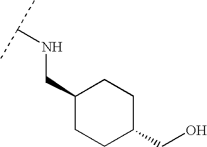 | NO₂ | CH₃ | 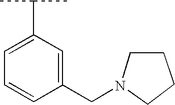 | C | 546(M + H)⁺ |
| 50 | 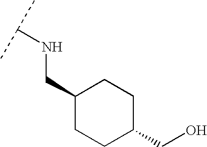 | NO₂ | CH₃ | 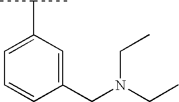 | C | 548(M + H)⁺ |
| 51 | 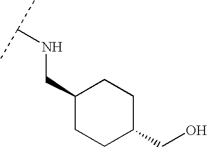 | NO₂ | CH₃ | 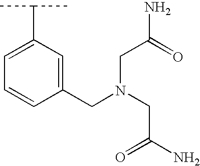 | C | 606(M + H)⁺ |
| 52 | 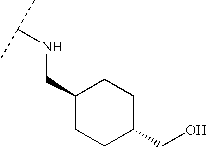 | NO₂ | CH₃ | 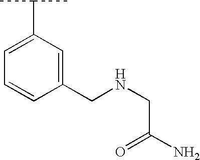 | C | 549(M + H)⁺ |
| 53 | 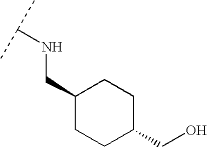 | NO₂ | Cl | 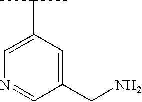 | C | 512(M + H)⁺ |
| 54 | 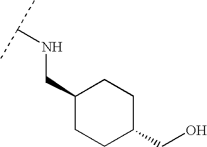 | NO₂ | OCF₃ | 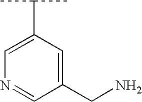 | C | 562(M + H)⁺ |
| 55 | 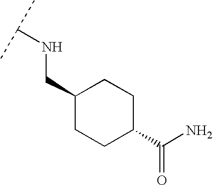 | NO₂ | CH₃ | 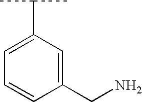 | C | 504(M + H)⁺ |
| 56 | 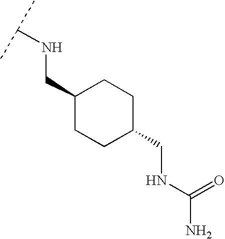 | NO₂ | CH₃ | 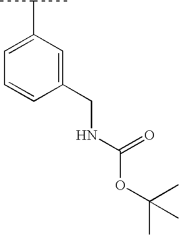 | C | 631(M + H)⁺ |
| 57 | 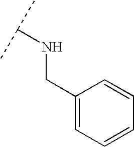 | NO₂ | CH₃ | 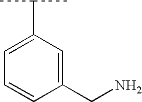 | C | 455(M + H)⁺ |
| 58 | 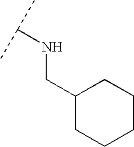 | NO₂ | CH₃ | 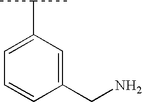 | C | 462(M + H)⁺ |
| 59 | 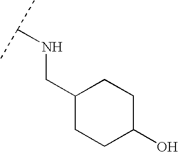 | NO₂ | CH₃ | 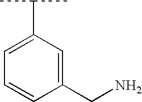 | C | 477(M + H)¹ |
| 60 | 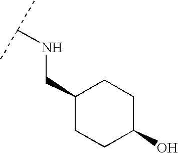 | NO₂ | CH₃ | 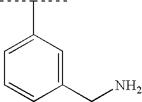 | C | 478(M + H)⁺ |
| 61 | 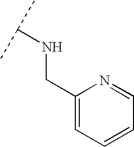 | NO₂ | CH₃ | 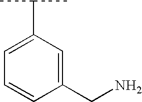 | C | 456(M + H)⁺ |
| 62 | 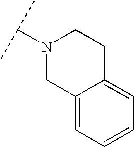 | NO₂ | CH₃ | 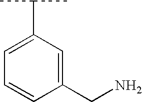 | C | 482(M + H)⁺ |
| 63 | 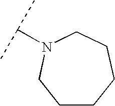 | NO₂ | CH₃ | 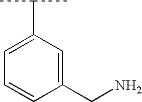 | C | 448(M + H)⁺ |
| 64 | 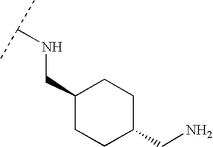 | NO₂ | Cl | 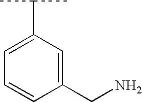 | C | 511(M + H)⁺ |
| 65 | 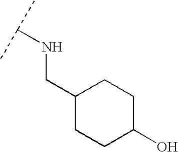 | NO₂ | Cl | 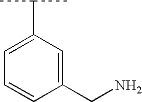 | C | 498(M + H)⁺ |
| 66 | 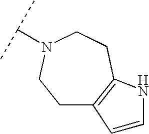 | NO₂ | Cl | 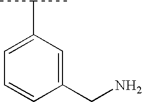 | C | 506(M + H)⁺ |
| 67 | 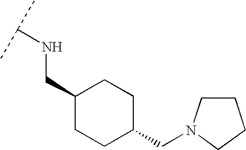 | NO₂ | Cl | 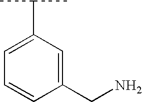 | C | 565(M + H)⁺ |
| 68 | 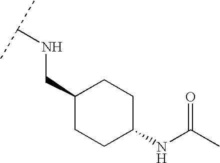 | NO₂ | H | 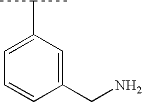 | C | 504(M + H)⁺ |
| 69 | 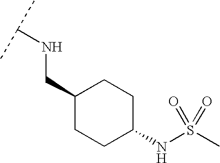 | NO₂ | CH₃ | 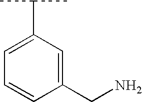 | C | 555(M + H)⁺ |
| 70 | 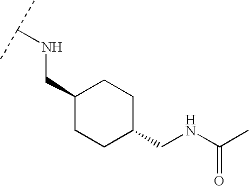 | NO₂ | CH₃ | 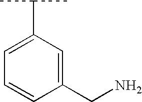 | C | 533(M + H)⁺ |
| 71 | 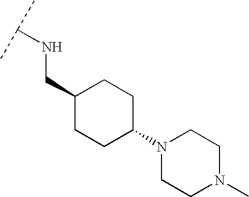 | NO₂ | CH₃ | 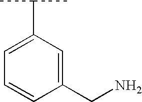 | C | 559(M + H)⁺ |
| 72 | 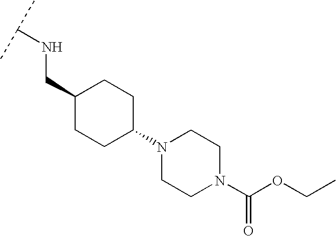 | NO₂ | CH₃ | 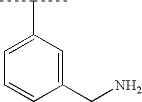 | C | 617(M + H)⁺ |
| 73 | 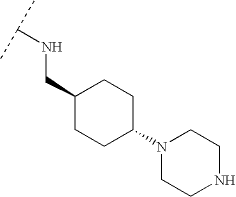 | NO₂ | CH₃ | 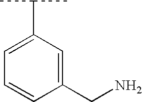 | C | 546(M + H)⁺ |
| 74 | 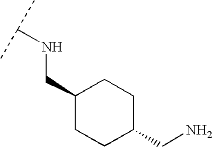 | NO₂ | CH₃ | 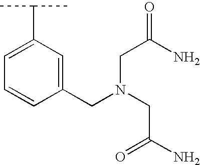 | C | 604(M + H)⁺ |
| 75 | 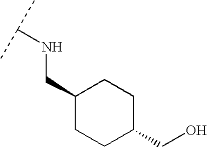 | CN | CH₃ | 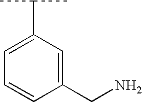 | C | 471(M + H)⁺ |

### Assessment of Biological Activity

### PKC-theta Inhibition Assay

The ability of compounds to inhibit the kinase activity of PKC-theta was measured using a firefly-luciferase reagent (PKLight™-Cambrex #LT07).

Compounds are diluted in 100% DMSO at 100× the final desired assay concentration.

Compounds are subsequently diluted 1:25 into complete assay buffer (50 mM HEPES/KOH, pH 7.5; 10 mM MgCl₂; 50 mM KCl; 0.01% CHAPS; 0.1% BSA; 200 μM TCEP). 25 μl of the 4× in 4% DMSO stocks are transferred to 384-well white polystyrene plates (Greiner #781075). 25 μl of a mixture containing 20 KM peptide substrate and 4 μM ATP are added to the compounds; followed by 50 μl of 4 nM PKC-theta. Blank wells are defined by the addition of an equal volume of assay buffer in place of the PKC-theta. Final assay concentrations are as follows: 2 nM PKC-theta, 5 μM peptide substrate, 1 μM ATP. The complete reaction is allowed to incubate at room temperature for 90-120 minutes. Following this incubation period the reaction is terminated by the addition of 100 μl of the PKLight™ reagent. This reaction is allowed to incubate for 15 minutes after which luminescence is quantified using an LJL Analyst.

The compounds in the synthetic examples above that were evaluated in the firefly-luciferase PKC-theta assay above were found to have IC₅₀'s less than 1 microM; preferred compounds had IC₅₀'s equal to or less than 0.050 microM. Some of the compounds in the synthetic examples above were also tested against Syk, Lyn, Veg-f and insulin receptor kinase to evaluate selectivity for PKC-theta inhibition. Some compounds were also tested against other kinases including CDK-2 and PLK. Many of the compounds demonstrated selectivity for the inhibition of PKC-theta as compared to one or more of the other kinases tested.

Assay conditions for testing against other kinases are generally known in the art.

Examples of suitable assays that can be used are described below

### SYK Kinase Assay

Syk is purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu4: Tyrl (PGTYR).

The kinase assay is performed in kinase assay buffer (50 mM HEPES, pH 7.0, 25 mM MgCl₂, 5 mM MnCl₂, 50 mM KCl, 100 μM Na3VO4, 0.2% BSA, 0.01% CHAPS, 200 μM TCEP). Test compounds initially dissolved in DMSO at 5 mg/mL, are pre-diluted for dose response (starting conc. 10 μM (or 5 μg/mL), 1 to 3 serial dilutions, 10 doses) with the assay buffer in 96-well polypropylene microtiter plates. A 40 μL volume of diluted enzyme (0.5 nM final conc.) in kinase buffer and a 20 μL aliquot of diluted compound are sequentially added to neutravidin coated 96-well white plate (PIERCE). The kinase reaction is started with a 40 μL volume of a mixture of substrates containing 0.75 μM ATP plus 4.5 ng/μL PGTYR-biotin (CIS Biointernational) in kinase buffer. Background wells are incubated with kinase plus buffer, and the reference inhibitor wells are incubated with 20 μL of 25 μM ADP instead of the compound. The assay plates are incubated for 30 min at room temperature. Following incubation, the assay plates are washed three times with 250 μL wash buffer (50 mM Tris-HCL, pH 7.4, 150 mM NaCl, 0.05% Tween 20, 0.2% BSA). A 100 μL aliquot of europium-labeled anti-phosphotyrosine (Eu3+-PT66, Wallac CR04-100) diluted in 50 mM Tris-HCl, pH 7.8, 150 mM NaCl, 10 μM DTPA, 0.05% Tween 40, 0.2% BSA, 0.05% BGG (1 nM final conc.) is added to each well and incubated for 30 min at room temperature. Upon completion of the incubation, the plate is washed four times with 250 μL of wash buffer and 100 μL of DELFIA Enhancement Solution (Wallac) is added to each well. After 15 min or longer, time-resolved fluorescence is measured on the LJL's Analyst (excitation at 360 nm, emission at 620 nm, EU 400 Dichroic Mirror) after a delay time of 250 μs.

### LYN Kinase Assay

Lyn(Kd) is purified as a GST-fusion protein. The kinase activity is measured using DELFIA (Dissociation Enhanced Lanthanide Fluoroimmunoassay) which utilizes europium chelate-labeled anti-phosphotyrosine antibodies to detect phosphate transfer to a random polymer, poly Glu4: Tyrl (PGTYR).

The kinase assay is performed in kinase assay buffer (50 mM HEPES, pH 7.0, 25 mM MgCl₂, 5 mM MnCl₂, 50 mM KCl, 100 μM Na₃VO₄, 0.2% BSA, 0.01% CHAPS, 200 μM TCEP). Test compounds initially dissolved in DMSO at 5 mg/mL, are pre-diluted for dose response (starting conc. 10 μM (or 5 μg/mL), 1 to 3 serial dilutions, 10 doses) with the assay buffer in 96-well polypropylene microtiter plates. A 40 μL volume of diluted enzyme (0.7 nM final conc.) in kinase buffer and a 20 μL aliquot of diluted compound are sequentially added to neutravidin coated 96-well white plate (PIERCE). The kinase reaction is started with a 40 μL volume of a mixture of substrates containing 1.25 μM ATP plus 4.5 ng/μL PGTYR-biotin (CIS Biointernational) in kinase buffer. Background wells are incubated with kinase plus buffer, and the reference inhibitor wells are incubated with 20 μL of 25 μM ADP instead of the compound. The assay plates are incubated for 30 min at room temperature. Following incubation, the assay plates are washed three times with 250 μL wash buffer (50 mM Tris-HCL, pH 7.4, 150 mM NaCl, 0.05% Tween 20, 0.2% BSA). A 100 μL aliquot of europium-labeled anti-phosphotyrosine (Eu3+-PT66, Wallac CR04-100) diluted in 50 mM Tris-HCl, pH 7.8, 150 mM NaCl, 10 μM DTPA, 0.05% Tween 40, 0.2% BSA, 0.05% BGG (1 nM final conc.) is added to each well and incubated for 30 min at room temperature. Upon completion of the incubation, the plate is washed four times with 250 μL of wash buffer and 100 μL of DELFIA Enhancement Solution (Wallac) is added to each well. After 15 min or longer, time-resolved fluorescence is measured on the LJL's Analyst (excitation at 360 nm, emission at 620 nm, EU 400 Dichroic Mirror) after a delay time of 250 μs.

### Methods of Therapeutic Use

The compounds of the invention are effective inhibitors of PKC-theta activity, and therefore are useful to inhibit PKC-theta activity in a patient and treat a variety of diseases and disorders that are mediated or sustained through the activity of PKC-theta. Without wishing to be bound by theory, the compounds of this invention would be expected to inhibit T cell activation via effective inhibition of PKC-theta, and are therefore useful to treat diseases and disorders associated with T cell activation. For example, the inhibition of T cell activation is therapeutically useful for selectively suppressing the immune function. Thus, the inhibition of PKC-theta with the compounds of this invention is an attractive means for treating a variety of immunological disorders, including inflammatory diseases, autoimmune diseases, organ and bone marrow transplant rejection and other disorders associated with T cell mediated immune response. In particular, the compounds of the invention may be used to treat acute or chronic inflammation, allergies, contact dermatitis, psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection) and lupus crythematosus. Other disorders associated with T cell-mediated immunc responses will be evident to those of ordinary skill in the art and can also be treated with the compounds and compositions of this invention.

In addition, PKC theta activation has been shown to be associated with insulin resistance in skeletal muscle. Therefore, the inhibition of PKC-theta with the compounds of this invention is also an attractive means for treating type II diabetes.

For therapeutic use, the compounds of the invention may be administered via a pharmaceutical composition in any conventional pharmaceutical dosage form in any conventional manner. Conventional dosage forms typically include a pharmaceutically acceptable carrier suitable to the particular dosage form selected. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compounds of this invention may be administered alone or in combination with adjuvants that enhance stability of the inhibitors, facilitate administration of pharmaceutical compositions containing them in certain embodiments, provide increased dissolution or dispersion, increase inhibitory activity, provide adjunct therapy, and the like, including other active ingredients. In one embodiment, for example, multiple compounds of the present invention can be administered. Advantageously, such combination therapies utilize lower dosages of the conventional therapeutics, thus avoiding possible toxicity and adverse side effects incurred when those agents are used as monotherapies. Compounds of the invention may be physically combined with the conventional therapeutics or other adjuvants into a single pharmaceutical composition. Advantageously, the compounds may then be administered together in a single dosage form. In some embodiments, the pharmaceutical compositions comprising such combinations of compounds contain at least about 5%, but more preferably at least about 20%, of a compound of formula (I) (w/w) or a combination thereof. The optimum percentage (w/w) of a compound of the invention may vary and is within the purview of those skilled in the art. Alternatively, the compounds of the present invention and the conventional therapeutics or other adjuvants may be administered separately (either scrially or in parallel). Separate dosing allows for greater flexibility in the dosing regime.

As mentioned above, dosage forms of the compounds of this invention may include pharmaceutically acceptable carriers and adjuvants known to those of ordinary skill in the art and suitable to the dosage form. These carriers and adjuvants include, for example, ion exchangers, alumina, aluminum stearate, lecithin, serum proteins, buffer substances, water, salts or electrolytes and cellulose-based substances. Preferred dosage forms include tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements for the compounds of the present invention may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

1. A compound having the following formula (I): 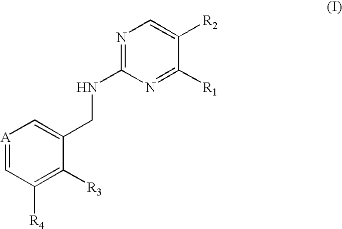
wherein:
R₁ is selected from the following groups: 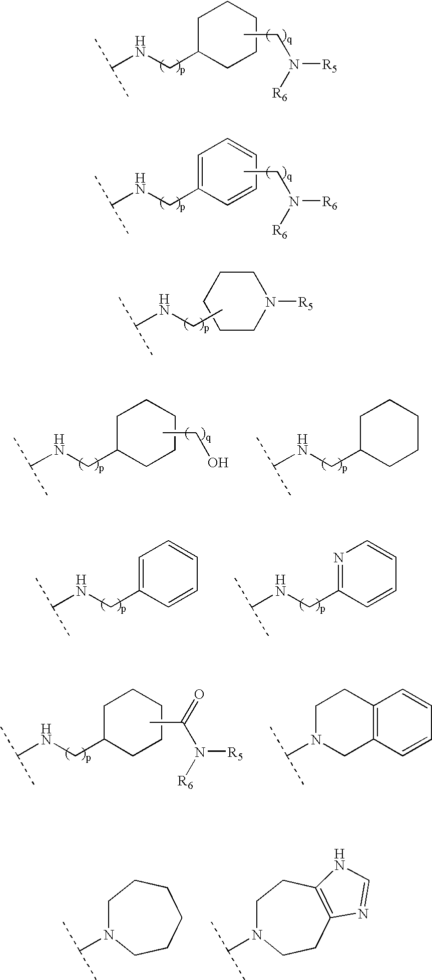
wherein:
p is 1, 2 or 3;
q is 0 or 1,
R₅, R₆ are each independently selected from:
(A) hydrogen,
(B) C₁₋₆alkyl, or wherein R₅ and R₆ together constitute a methylene bridge which together with the nitrogen atom between them forms a four to six-membered ring wherein one of the methylene groups is optionally replaced by an oxygen or nitrogen atom, and which ring is optionally and independently substituted by one or more of the following groups:
(i) C₁₋₆alkyl
(ii) COR₇, wherein R₇ is:
(a) C₁₋₆alkyl,
(b) C₁₋₆alkyloxy,
(C) C₁₋₆alkylcarbonyl,
(D) C₁₋₆alkylsulfonyl,
(E)-CONR₈R₉, wherein R₈ and R₉ are each independently selected from:
(i) hydrogen
(ii) C₁₋₆alkyl;
R₂ is selected from the following groups:
(A) CF₃,
(B) cyano,
(C) CONH₂
(D) halogen, or
(E) nitro;
R₃ is selected from the following groups:
(A) hydrogen,
(B) C₁₋₆alkyl, which is optionally substituted with halogen,
(C) C₁₋₆alkyloxy, which is optionally substituted with halogen,
(D) halogen,
R₄ is selected from the following groups:
(A) heteroaryl, which is optionally substituted with C₁₋₆alkyl;
(B) aryl or heteroaryl, which is substituted with one or more of the following groups:
(i) C₁₋₆alkyl, which is substituted with hydroxyl, oxo, or NR₁₀R₁₁, wherein R₁₀ and R₁₁ are each independently selected from the following groups:
(a) hydrogen,
(b) C₁₋₆alkyl, which is optionally substituted with hydroxyl or CONH₂,
(c) C₁₋₆alkylcarbonyl, which is optionally substituted with one or more halogens,
(d) C₁₋₆alkylsulfonyl,
(e) or wherein R₁₀ and R₁₁ constitute a methylene bridge which together with the nitrogen atom between them forms a four to six-membered ring,
(ii) CONR₁₂R₁₃, wherein R₁₂ and R₁₃ are each independently selected from hydrogen or C₁₋₆alkyl,
(iii) SO₂NR₁₂R₁₃, wherein R₁₂ and R₁₃ are each independently selected from hydrogen or C₁₋₆alkyl,
(C) —NR₁₄R₁₅, wherein R₁₄ and R₁₅ are each independently selected from:
(i) C₁₋₆alkylcarbonyl, which is substituted with amino,
(ii) or wherein R₁₄ and R₁₅ constitute a methylene bridge which together with the nitrogen atom between them forms a four to seven-membered ring, wherein one of the methylene groups is substituted with C₁₋₆alkyl, and wherein each C₁₋₆alkyl is optionally substituted with hydroxyl or NR₁₀R₁₁, wherein R₁₀ and R₁₁ are as defined previously,
(D) —CONR₁₆R₁₇, wherein R₁₆ and R₁₇ are each independently selected from:
(i) C₁₋₆alkyl, which is substituted with hydroxyl or NR₁₈R₁₉, wherein R₁₈ and R₁₉ are each independently selected from hydrogen or C₁₋₆alkyl, or wherein R₁₈ and R₁₉ constitute a methylene bridge which together with the nitrogen atom between them forms a four to six-membered ring, wherein one of the methylene groups is optionally replaced by an oxygen;
(E) C₁₋₆alkynyl group optionally substituted by amino, C₁₋₃alkylamino, or di-(C₁₋₃alkyl)amino; and
A is independently selected from carbon or nitrogen;
or a tautomer, pharmaceutically acceptable salt, solvate or amino-protected derivative thereof.

2. A compound according to claim 1, wherein:
R₁ is selected from the following groups: 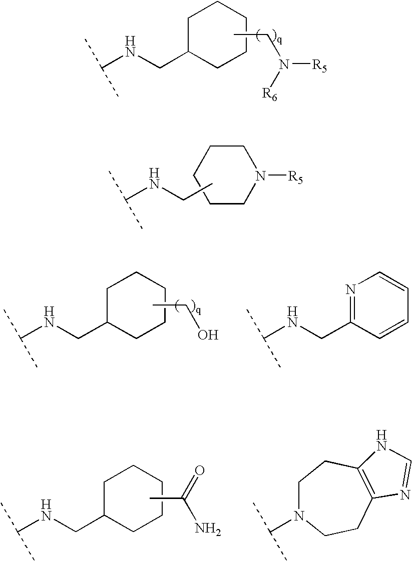
wherein:
q is 0 or 1,
R₅, R₆ are each independently selected from:
(A) hydrogen,
(B) or wherein R₅ and R₆ together constitute a methylene bridge which together with the nitrogen atom between them forms a five to six-membered ring wherein one of the methylene groups is optionally replaced by a nitrogen atom, and which ring is optionally and independently substituted by one or more of the following groups:
(i) C₁₋₆alkyl
(ii) COR₇, wherein R₇ is C₁₋₆alkyloxy,
(C) C₁₋₆alkylcarbonyl
(D) C₁₋₆alkylsulfonyl;
R₂ is selected from the following groups:
(A) cyano, or
(B) nitro;
R₃ is selected from the following groups:
(A) C₁₋₃alkyl,
(B) C₁₋₃alkyloxy, which is optionally substituted with fluorine,
(C) halogen;
R₄ is selected from the following groups:
(A) aryl, which is substituted with one or more of the following groups:
(i) C₁₋₃alkyl, which is substituted with hydroxyl or NR₂₀R₂₁, wherein R₂₀ and R₂₁ are each independently selected from the following groups:
(a) hydrogen,
(b) C₁₋₃alkyl, which is optionally substituted with hydroxyl or CONH₂,
(c) or wherein R₂₀ and R₂₁ constitute a methylene bridge which together with the nitrogen atom between them forms a five to six-membered ring,
(ii) CONH₂
(iii) SO₂NH₂,
(B) 3-pyridyl, which is optionally substituted with C₁₋₃alkyl, wherein each alkyl group is optionally substituted with amino,
(C)—NR₂₂R₂₃, wherein R₂₂ and R₂₃ constitute a methylene bridge which together with the nitrogen atom between them forms a five to six-membered ring, wherein one of the methylene groups is substituted with C₁₋₃alkyl, and wherein each C₁₋₃alkyl is optionally substituted with OH or NR₂₀R₂₁, where R₂₀ and R₂₁ are as defined previously,
(D)-CONR₂₄R₂₅, wherein R₂₄ and R₂₅ are each independently selected from:
(iv) C₁₋₃alkyl, which is substituted with C₁₋₃alkylamino; and
A is independently selected from carbon or nitrogen;
or a tautomer, pharmaceutically acceptable salt, solvate or amino-protected derivative thereof.

3. A compound according to claim 1, having the following formula (II): wherein: 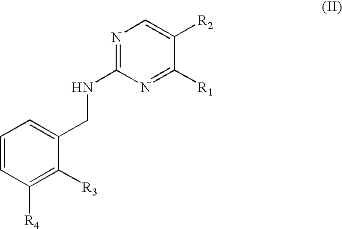
R₁ is selected from the following groups: 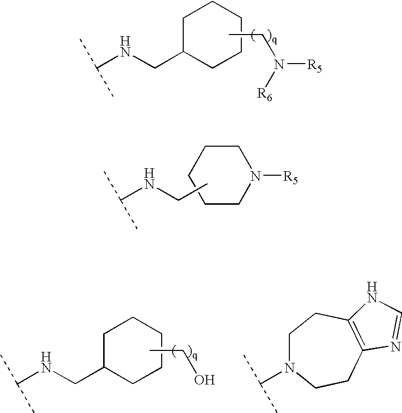
wherein:
q is 0 or 1
R₅, R₆ are each independently selected from:
(A) hydrogen,
(B) C₁₋₆alkylcarbonyl,
(C) C₁₋₆alkylsulfonyl;
R₂ is selected from the following groups:
(A) cyano, or
(B) nitro;
R₃ is selected from the following groups:
(A) CH₃,
(B) OCF₃,
(C) Cl;
R₄ is selected from the following groups: 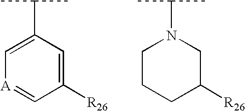
wherein:
R₂₆ is selected from the following groups:
(A) C₁₋₃alkyl, which is substituted with hydroxyl or NR₂₇R₂₈, wherein R₂₇ and R₂₈ are each independently selected from the following groups:
(i) hydrogen,
(ii) C₁₋₃alkyl, which is optionally substituted with hydroxyl or CONH₂,
(B) CONH₂
(C)SO₂NH₂; and
A is carbon or nitrogen;
or a tautomer, pharmaceutically acceptable salt, solvate or amino-protected derivative thereof.

4. A compound according to claim 1, selected from the compounds in the following table:

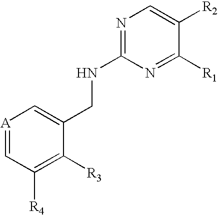

| Ex | | | | | |
|---|---|---|---|---|---|
| # | R₁ | R₂ | R₃ | R₄ | A |
| 1 | 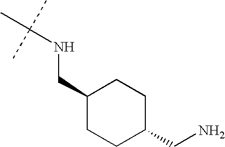 | NO₂ | H | 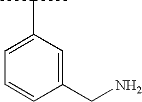 | C |
| 2 | 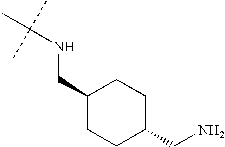 | NO₂ | H | 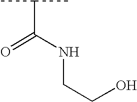 | C |
| 3 | 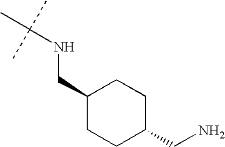 | NO₂ | H | 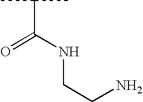 | C |
| 4 | 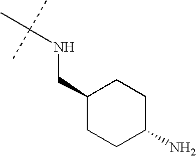 | NO₂ | CH₃ | 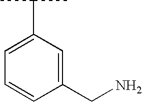 | C |
| 5 | 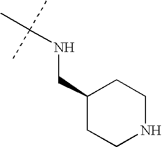 | NO₂ | CH₃ | 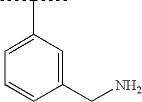 | C |
| 6 | 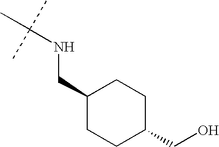 | NO₂ | H | 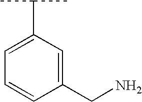 | C |
| 7 | 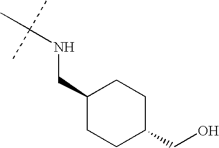 | NO₂ | H | 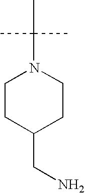 | C |
| 8 | 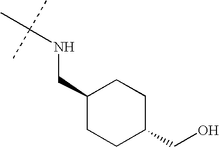 | NO₂ | H | 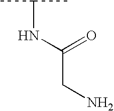 | C |
| 9 | 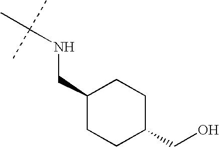 | NO₂ | CH₃ | 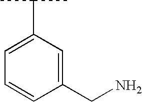 | C |
| 10 | 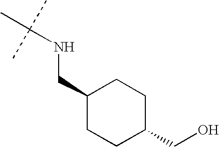 | NO₂ | CH₃ | 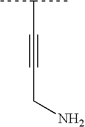 | C |
| 11 | 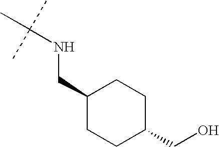 | NO₂ | CH₃ | 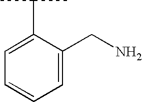 | C |
| 12 | 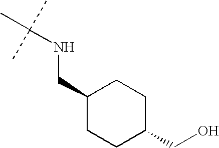 | NO₂ | CH₃ | 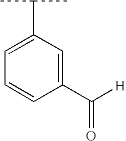 | C |
| 13 | 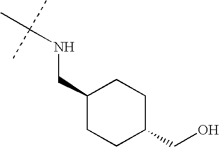 | NO₂ | CH₃ | 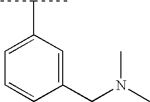 | C |
| 14 | 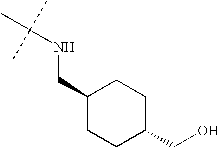 | NO₂ | CH₃ | 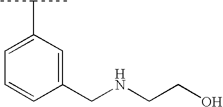 | C |
| 15 | 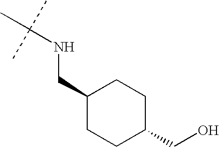 | NO₂ | CH₃ | 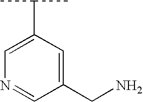 | C |
| 16 | 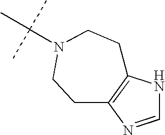 | NO₂ | CH₃ | 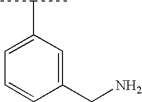 | C |
| 17 | 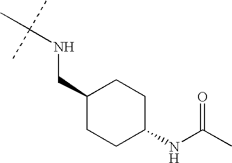 | NO₂ | CH₃ | 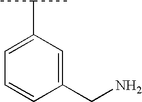 | C |
| 18 | 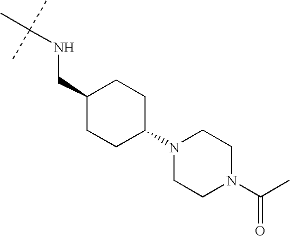 | NO₂ | CH₃ | 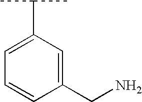 | C |
| 19 | 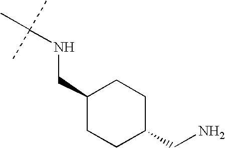 | NO₂ | CH₃ | 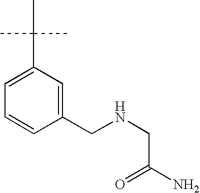 | C |
| 20 | 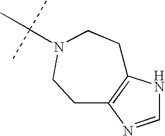 | CN | CH₃ | 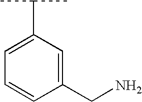 | C |
| 21 | 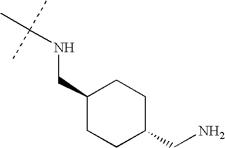 | NO₂ | H | 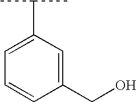 | C |
| 22 | 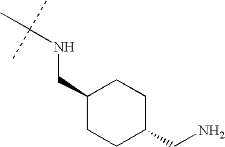 | NO₂ | H | 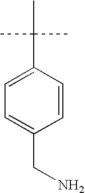 | C |
| 23 | 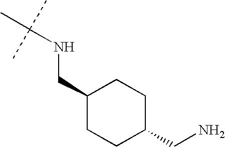 | NO₂ | H | 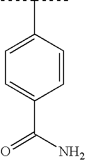 | C |
| 24 | 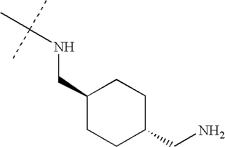 | NO₂ | H | 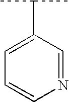 | C |
| 25 | 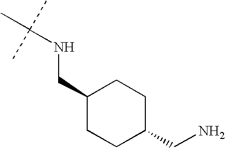 | NO₂ | H | 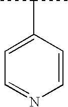 | C |
| 26 | 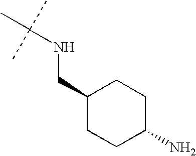 | NO₂ | H | 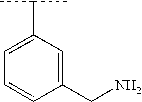 | C |
| 27 | 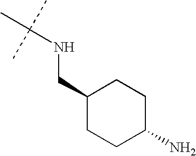 | NO₂ | H | 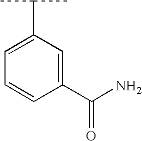 | N |
| 28 | 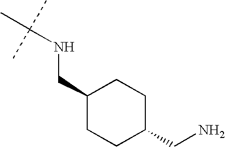 | NO₂ | H | 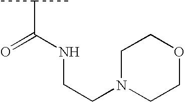 | C |
| 29 | 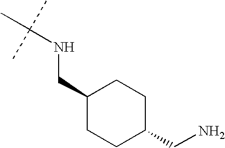 | NO₂ | H | 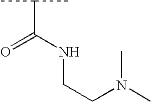 | C |
| 30 | 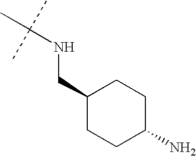 | NO₂ | Cl | 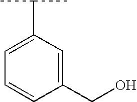 | C |
| 31 | 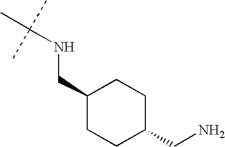 | NO₂ | CH₃ | 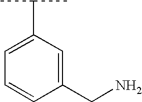 | C |
| 32 | 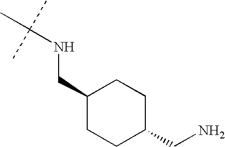 | NO₂ | CH₃ | 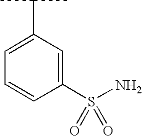 | C |
| 33 | 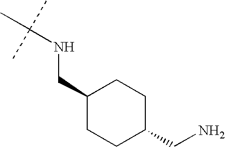 | NO₂ | CH₃ | 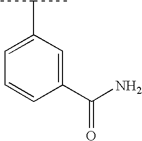 | C |
| 34 | 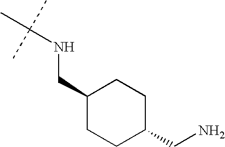 | NO₂ | CH₃ | 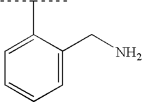 | C |
| 35 | 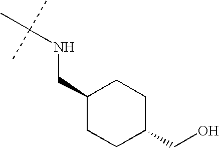 | NO₂ | H | 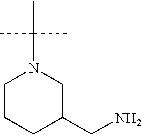 | C |
| 36 | 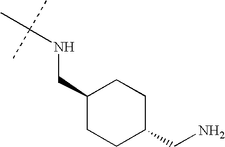 | NO₂ | H | 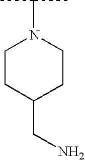 | C |
| 37 | 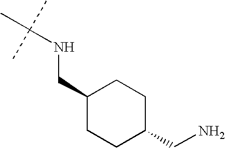 | NO₂ | H | 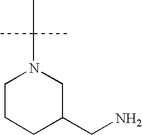 | C |
| 38 | 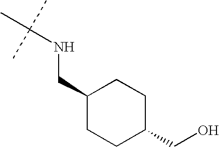 | NO₂ | H | 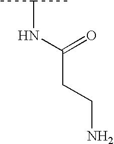 | C |
| 39 | 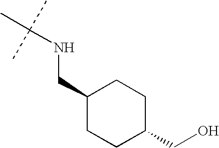 | NO₂ | CH₃ | 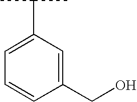 | C |
| 40 | 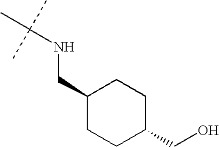 | NO₂ | CH₃ | 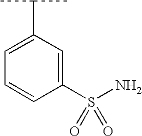 | C |
| 41 | 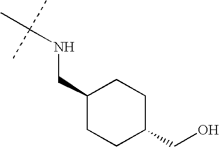 | NO₂ | CH₃ | 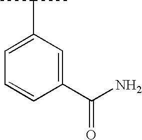 | C |
| 42 | 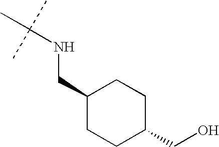 | NO₂ | CH₃ | 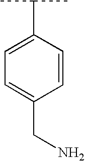 | C |
| 43 | 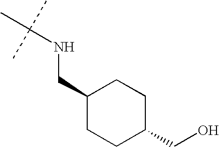 | NO₂ | CH₃ | 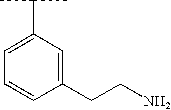 | C |
| 44 | 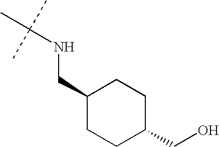 | NO₂ | F | 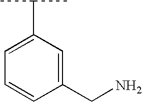 | C |
| 45 | 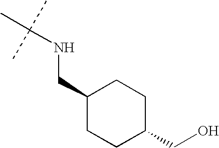 | NO₂ | OCF₃ | 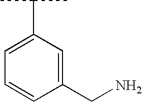 | C |
| 46 | 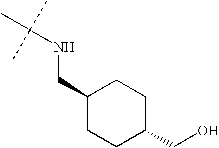 | NO₂ | Cl | 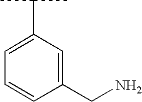 | C |
| 47 | 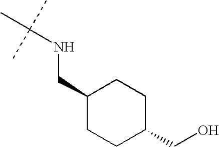 | NO₂ | CH₃ | 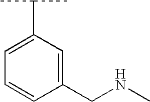 | C |
| 48 | 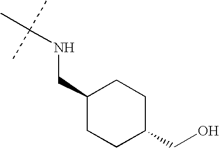 | NO₂ | CH₃ | 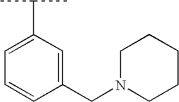 | C |
| 49 | 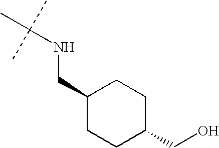 | NO₂ | CH₃ | 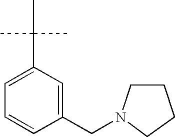 | C |
| 50 | 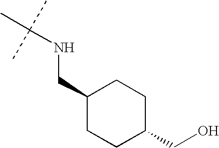 | NO₂ | CH₃ | 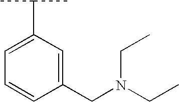 | C |
| 51 | 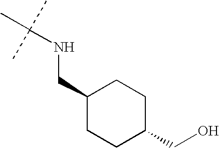 | NO₂ | CH₃ | 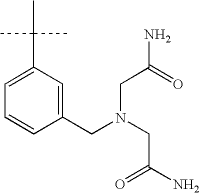 | C |
| 52 | 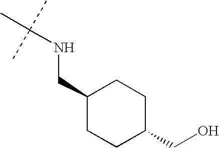 | NO₂ | CH₃ | 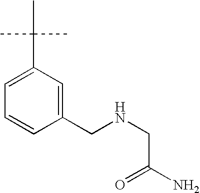 | C |
| 53 | 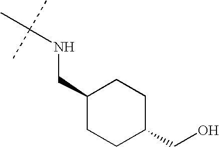 | NO₂ | Cl | 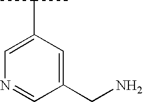 | C |
| 54 | 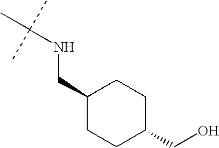 | NO₂ | OCF₃ | 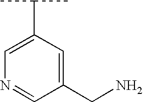 | C |
| 55 | 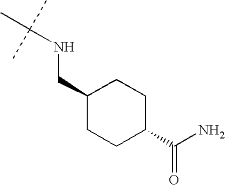 | NO₂ | CH₃ | 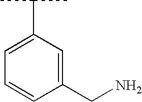 | C |
| 56 | 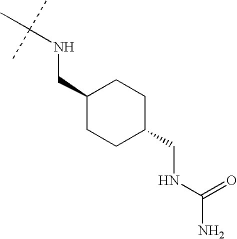 | NO₂ | CH₃ | 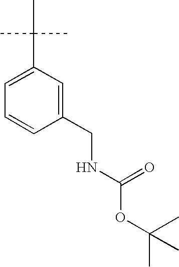 | C |
| 57 | 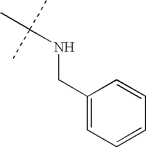 | NO₂ | CH₃ | 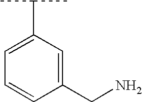 | C |
| 58 | 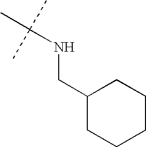 | NO₂ | CH₃ | 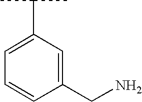 | C |
| 59 | 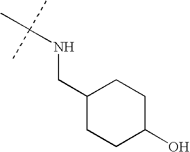 | NO₂ | CH₃ | 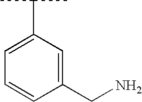 | C |
| 60 | 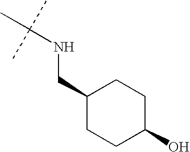 | NO₂ | CH₃ | 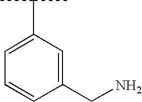 | C |
| 61 | 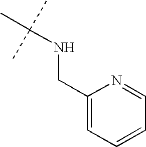 | NO₂ | CH₃ | 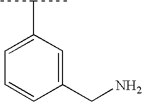 | C |
| 62 | 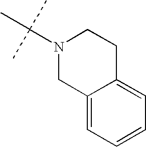 | NO₂ | CH₃ | 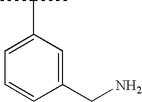 | C |
| 63 | 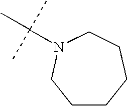 | NO₂ | CH₃ | 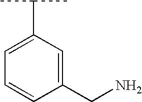 | C |
| 64 | 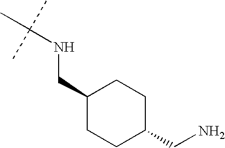 | NO₂ | Cl | 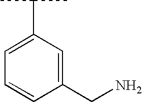 | C |
| 65 | 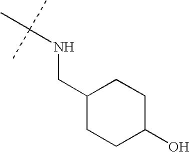 | NO₂ | Cl | 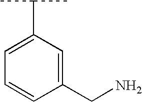 | C |
| 66 | 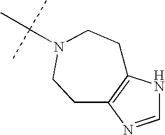 | NO₂ | Cl | 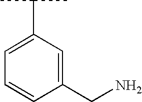 | C |
| 67 | 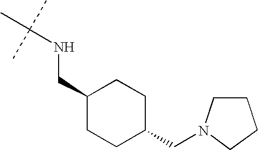 | NO₂ | Cl | 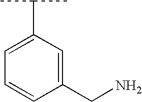 | C |
| 68 | 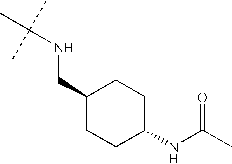 | NO₂ | H | 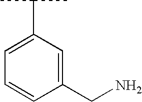 | C |
| 69 | 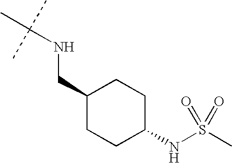 | NO₂ | CH₃ | 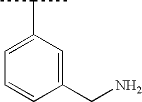 | C |
| 70 | 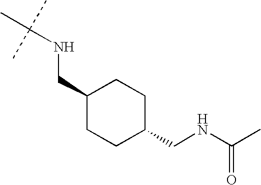 | NO₂ | CH₃ | 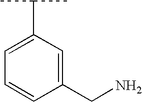 | C |
| 71 | 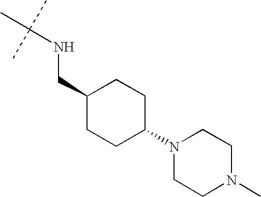 | NO₂ | CH₃ | 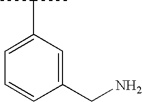 | C |
| 72 | 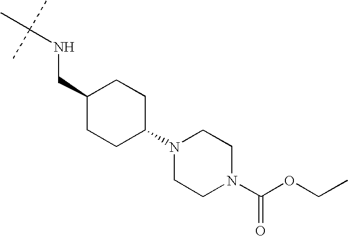 | NO₂ | CH₃ | 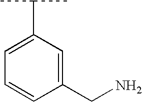 | C |
| 73 | 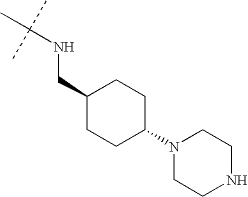 | NO₂ | CH₃ | 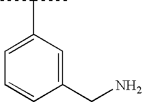 | C |
| 74 | 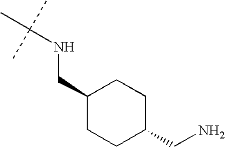 | NO₂ | CH₃ | 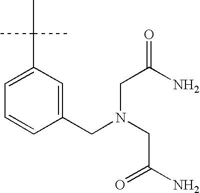 | C |
| 75 | 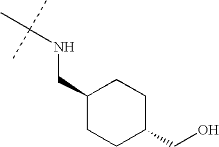 | CN | CH₃ | 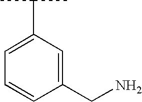 | C |

5. (canceled)

6. A pharmaceutical composition comprising a compound according to claim 1, and at least one pharmaceutically acceptable carrier or adjuvant.

7. A method for treating a disease or disorder that is mediated or sustained through the activity of PKC-theta in a patient comprising administering to said patient a therapeutically effective amount of a compound according to claim 1.

8. A method for treating a disease or disorder associated with the activation of T cells in a patient comprising administering to said patient a therapeutically effective amount of a compound according to claim 1.

9. A method for treating an immunological disorder, an inflammatory disease, an autoimmune disease, organ and bone marrow transplant rejection, acute or chronic inflammation, allergies, contact dermatitis, psoriasis, rheumatoid arthritis, multiple sclerosis, type I diabetes, inflammatory bowel disease, Guillain-Barre syndrome, Crohn's disease, ulcerative colitis, graft versus host disease, lupus erythematosus or type II diabetes in a patient comprising administering to said patient a therapeutically effective amount of a compound according to claim 1.
